(19) United States
(12) Patent Application Publication
(10) Pub. No.: US 2025/0333708 A1
(43) Pub. Date: Oct. 30, 2025

(54) MANIPULATING NEPHRON DIFFERENTIATION RATE IN INDUCED HUMAN PLURIPOTENT STEM CELL ORGANOIDS AND TISSUES BY ENGINEERING MECHANICS OF THE MICROENVIRONMENT

(71) Applicant: THE TRUSTEES OF THE UNIVERSITY OF PENNSYLVANIA, Philadelphia, PA (US)

(72) Inventors: Alex HUGHES, Philadelphia, PA (US); John VIOLA, Philadelphia, PA (US); Jiageng LIU, Philadelphia, PA (US); Louis S. PRAHL, Philadelphia, PA (US); Zheyuan HUANG, Philadelphia, PA (US); Catherine PORTER, Philadelphia, PA (US)

(21) Appl. No.: 18/868,301

(22) PCT Filed: Jun. 2, 2023
(86) PCT No.: PCT/US2023/067804

§ 371 Date: Nov. 22, 2024

Related U.S. Application Data
(60) Provisional application No. 63/365,790, filed on Jun. 3, 2022.

(51) Int. Cl. C12N 5/071; C12M 1/42

(52) U.S. Cl. CPC C12N 5/0686; C12M 35/04; C12N 5/0687; C12N 2501/155; C12N 2501/415; C12N 2501/727; C12N 2513/00; C12N 2527/00

(57) ABSTRACT

Provided are methods to increase the nephron yield and control over nephron locations in kidney organoids and kidney replacement tissues; the methods achieve spatiotemporal control over the mechanical microenvironment to engineer favorable environments for nephron formation within kidney organoids. This engineered control can be created using microdevices that impose mechanical stress at defined intervals synchronized with cyclical nephron development in vivo, agonists/antagonists of tension-generating biochemical pathways in whole organoids or cells, optogenetic control over tension in stem cell collectives, and/or cell-cell junction mediated transfer of mechanical information between cells, or similar approaches.

### CROSS-REFERENCE TO RELATED APPLICATIONS

The present application claims priority to and the benefit of U.S. patent application No. 63/365,790, “Manipulating Nephron Differentiation Rate In Induced Human Pluripotent Stem Cell Organoids And Tissues By Engineering Mechanics Of The Microenvironment” (filed Jun. 3, 2022). All foregoing applications are incorporated herein by reference in their entireties for any and all purposes.

### GOVERNMENT RIGHTS

This invention was made with government support under 2047271 awarded by the National Science Foundation and GM133380 and DK132296 awarded by the National Institutes of Health. The government has certain rights in the invention.

### TECHNICAL FIELD

The present disclosure relates to the field of cell differentiation and to the field of nephron cells.

### BACKGROUND

There has been some progress in recent years in the development of kidney organoids and tissue engineered kidneys, which would alleviate the need for human donor tissue in treating kidney disease. At the same time, challenges remain in achieving organoid maturity and higher order organization. Accordingly, there is a long-felt need in the art for improved methods of developing kidney organoids.

### SUMMARY

In meeting the described long-felt needs, the present disclosure provides a method of modulating nephron differentiation, comprising: effecting a change in mechanical stress experienced by at least one nephron progenitor cell so as to give rise to cell differentiation and primitive nephron aggregate formation by the at least one nephron progenitor cell, the at least one nephron progenitor cell optionally being present in a patterned arrangement.

Also provided is a system, the system configured to perform a method of the present disclosure (for example, according to any one of Aspects 1-19).

Further provided is a synthetic kidney comprising a nephron formed according to the method of the present disclosure (for example, according to any one of Aspects 1-19).

Additionally provided is a cartridge comprising a nephron formed according to the method of the present disclosure (for example, according to any one of Aspects 1-19).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the drawings, which are not necessarily drawn to scale, like numerals can describe similar components in different views. Like numerals having different letter suffixes can represent different instances of similar components. The drawings illustrate generally, by way of example, but not by way of limitation, various aspects discussed in the present document. In the drawings:

FIG. 1: Kidney curvature imposes topological requirements on geometry of close-packed ureteric tubules. (A) Confocal fluorescence micrograph showing maximum projection (top) and xz plane for embryonic day (E) 15 mouse kidney. Branching cytokeratin+ureteric epithelial tubule tips are surrounded by SIX2+nephron progenitors in the cap mesenchyme. Caps are separated by a thin layer of stroma (unlabeled). JAG1+ early nephrons form at ‘armpits’ beneath tubule tips. (B) Schematic of repulsive particle (radius a) packing at the surface of a sphere (radius R), showing pentagonal and heptagonal topological defects amongst an otherwise hexagonal packing. Defects form pairs, clusters, or chains for R/a>5. (C-F) Left, height maps of representative kidney surfaces over E14-E17 overlayed with ureteric tubule tip positions. Right, micrographs of combined ECAD and SIX2 immunofluorescence overlayed with Voronoi diagrams tracing ‘tip domains’. Voronoi cells are colored by coordination number (#neighbors). (G) Example Voronoi cells and corresponding tip domain boundaries. (H) Frequency plot of tip domain coordination number (for (H)-(J), n=5, 6, 4, 4 kidneys and 162, 353, 535, and 892 tip domains in E14, E15, E16, and E17 categories, respectively). (I) Average and standard deviation (s.d.) of coordination number distributions. (J) Sum of defect charge (i.e. net decrease in coordination number from z=6) plotted against integrated Gaussian curvature for patches of kidney surface (units of disclinations).

FIG. 2. Close packing of ureteric tips predicts a transition to rigidity and emergence of semi-crystallinity over developmental time. (A) Left, Segmented confocal micrograph indicating the area fraction ϕ of cap mesenchyme. Right, Plot of ϕ over developmental time relative to 2D square packing, random close packing (rcp), and hexagonal close packing (hcp) of circles (mean±S.D., n=3 kidneys per embryonic day, average over >12 tip domains per kidney). (B) Definition of shape index p of tip domains from Voronoi cell perimeter (P) and area (A). (C) Right, Isostaticity (degree of freedom) analysis to define rigidity threshold in terms of critical average coordination number zᵢₛₒ and critical shape index p* for the cell vertex model (left), and adapted to coupled tip domains (right), DOF=degrees of freedom. (D) Box plots of tip domain shape index distributions over time, relative to those for squares, pentagons (pent), and hexagons (hex), and to p* =3.91 (n =5, 6, 4, 4 kidneys and 177, 391, 617, and 987 tip domains in E14,E15, E16, and E17 categories, respectively. One-way ANOVA, Tukey's test: *p<0.05, **p<0.01, ***p<0.001). (E) Left column, Representative physical model output for tubule packing regimes characteristic of the indicated developmental ranges, previously reported in (17). Right column, overlays of Delauney triangulations indicating coordination number of tip domains on spatial heatmaps of shape index, showing reducing shape index as tip patterns transition from amorphous to square-like to hcp-like regimes. (F) Representative case study of amorphous and crystalline phases of tip domain packing at E17. Top, Confocal micrograph of tip domains and shape index heatmap. Example regions of different packing regimes are outlined in blue. Bottom, Inset of these regions showing lattice lines and decreasing median shape index p from amorphous to square-like to hcp-like packing. (G) Study of positional order within regions in (F) by spatial autocorrelation.

FIG. 3. The kidney surface becomes stiffer and less viscous over E15-17. (A) Schematic of surface microindentation using a cylindrical indenter that captures kidney viscoelastic properties on the length scale of ˜3 tip domains for h˜30 μm. (B) Cartoon of typical data output and key parameters during indentation run (left), and during subsequent recording of force at fixed final indentation depth (right). (C) Representative indentation force vs. indentation depth and force relaxation curves for E15-E17 kidneys. (D) Stiffness, relaxation time t, and viscous relaxation constant k plots over E15-E17 (mean±S.D., n>14 kidneys per embryonic day for stiffness and >8 for τ and k, one measurement per kidney. One-way ANOVA, Tukey's test: *p<0.05, **p<0.01, ***p<0.001). (E) Summary of changes in ureteric tip geometry and kidney surface mechanics over E15-17.

FIG. 4. Nephrogenesis rate varies over a tip domain ‘life-cycle’ defined by shape index. (A) Left, Cartoon showing side (xz) view appearance of a pair of ureteric tips associated with early stage (PTA, pre-tubular aggregate; RV, renal vesicle) and later stage (CSB, comma shaped body; SSB, S-shaped body) nephrons. Right, Confocal immunofluorescence micrographs at the mid-plane of ureteric tips and 20 um deeper into an E17 kidney. All nephrons associated with each tip can be annotated either as SIX2⁺ spheroids for early nephrons or from the attachment points of their connecting tubules for later nephrons (white circles at tips in PNA channel), see arrows. (B) Plot of tip domain area against shape index, with nephron number per tip in the color dimension (n=694 tip domains across 4 kidneys. One-way ANOVA, Tukey's test: *p<0.05, **p<0.01, ***p<0.001). Each axis is also plotted individually vs. nephron number per tip. (C) Cartoon of ureteric tip life-cycle and definition of a pseudotime based on shape index of tip domains. N.B. asynchronous tip branching is not depicted for clarity. (D) Example confocal micrographs of tip domains over a range in shape index. (E) Running average of nephron number per tip against shape index (window width=50 points).

FIG. 5. Newly established tip domains experience anisotropic stress that decreases over each life-cycle. (A) Schematic of inferred tension (T) and pressure (P) contributions to the force balance at an example node in a vertex model representation of tip domains. (B) Example force inference output from tip domain Voronoi geometry as a spatial map overlaid on confocal fluorescence micrograph of E17 kidney surface, along with relative principal and anisotropic stress vectors. (C) Box plots of isotropic and anisotropic stress for tip domains, relative to the median of the highest stress category in each plot (n=4 kidneys; 195, 319, and 164 tip domains in low, medium, and high shape index categories, respectively. One-way ANOVA, Tukey's test: *p<0.05, **p<0.01, ***p<0.001). (D) Top, Schematic of laser microdissection ablation of cap mesenchyme and stroma orthogonal to boundaries between neighboring tip domains at E17. Bottom, Confocal micrograph showing example cuts after fixation, cut location prior to ablation by widefield fluorescence, and rebound of the cut in the 45 s after ablation. (E) Sequential z-slices in a confocal stack after fixation, showing penetration of a cut ˜20 μm through the inter-tip region (cell integrity was lost down to ˜40 μm). (F) Plot of rebound velocity of cuts (change in cut width in 45 s) after ablation against the average shape index of the tip domains immediately adjacent to each cut (n=99 cuts pooled across 7 kidneys).

FIG. 6. Mimicking decrease in anisotropic stress increases nephron induction in human iPSC-derived kidney organoids. (A) Volcano plot of differentially expressed cytoskeleton and cell migration genes for committing vs. progenitor populations from scRNA-seq data in (24). Red circles are significantly differentially expressed genes. Coll., collagen. (B) Schematic of human iPSC differentiation to metanephric mesenchyme (met. mes.) and formation of spheroids for early nephron induction assays. Prim., primitive; PIM, posterior intermediate mesoderm. (C) Confocal immunofluorescence micrograph of iPSC-derived metanephric mesenchyme after continued 2D differentiation in FGF9 basal media until day 21, with a CHIR pulse over days 9-11. (D) Representative confocal immunofluorescence projections of spheroids after 48 hr perturbations. Inset, Detail of Jag1 expression. (E) Plot of marker positive pixel areas as a % of projection area for markers of nephron progenitors (SIX2) and early nephron cells (LHX1) (mean±S.D., n>4 spheroids pooled across two replicates. One-way ANOVA, Tukey's test: *p<0.05, **p<0.01, ***p<0.001). All conditions were comparable between replicates except for the +CHIR+Y27632 condition, where replicates (rep.) are reported as separate distributions. (F) Left, Confocal immunofluorescence of pSMAD1/5 in E17 kidney tip domains. Right, Box plot of mean cap mesenchyme pSMAD1/5 fluorescence of tip domains binned by their shape index (n=20, 76, and 37 tip domains pooled across 2 kidneys in low, medium, and high shape index categories, respectively. One-way ANOVA, Tukey's test: *p<0.05, **p<0.01, ***p<0.001). (G) Volcano plot of differentially expressed SMAD1 transcriptional targets for committing vs. progenitor populations from scRNA-seq data in (24). Red circles are significantly differentially expressed genes. (H) Schematic summary of curvature and packing contributions to tip domain geometry. (I) Schematic summary of cyclical mechanical stress in the tip domain microenvironment associated with tip duplication life-cycle, and hypothesis for mechanical licensing of differentiation signaling in NPCs.

FIG. 7. Kidney curvature analysis. (A) Perspective rendering (top row) and wireframes (bottom row) of example segmented E17 confocal stack and resulting surfaces generated after each processing step in Rhino (see Methods). (B) Midplane yz cross-sections of original segmented volume and loft surface after pre-processing steps, showing appropriate fit for subsequent curvature analysis. (C) Top (xy) view renderings of original volume, loft surface as a heatmap of height from MATLAB, and loft surface as a heatmap of Gaussian curvature from Rhino Grasshopper.

FIG. 8. Tip domains transition from rounding-dominated to area growth-dominated regimes over tip life-cycle. (A) A version of FIG. 4B divided into two periods during the tip life-cycle before (right side) and after (left side) nephrogenesis begins (FIG. 4E). Note that younger tip domains with fewer nephrons primarily round up prior to the nephrogenesis phase, which coincides with a higher rate of tip domain area increase. (B) A version of FIG. 4E except plotted against tip domain area rather than shape index.

FIG. 9. Gene-set enrichment analysis for committing vs. progenitor cell populations. (A) Top 10 gene sets enriched in committing vs. progenitor cell populations inferred from RNA-seq data in (24). (B) Enrichment plot for cell migration GO term.

FIG. 10. qPCR validates human iPSC differentiation through posterior intermediate mesoderm to metanephric mesenchyme. Plots of fold-change in OSR1 and PAX2 transcripts (see Methods) for human iPSCs differentiated over days 0-9 according to the Morizane protocol (66) (mean±S.D., n=3 wells per condition).

FIG. 11. pSMAD1/5 levels in caps do not correlate with tip domain area. Box plot of mean cap mesenchyme pSMAD1/5 fluorescence of tip domains binned by their area (n=20, 76, and 37 tip domains pooled across 2 kidneys in low, medium, and high area categories, respectively. One-way ANOVA, Tukey's test: *p<0.05, **p<0.01, ***p<0.001).

FIG. 12 provides an exemplary, non-limiting description of the relationship between mechanical stress and cell differentiation for nephron cells.

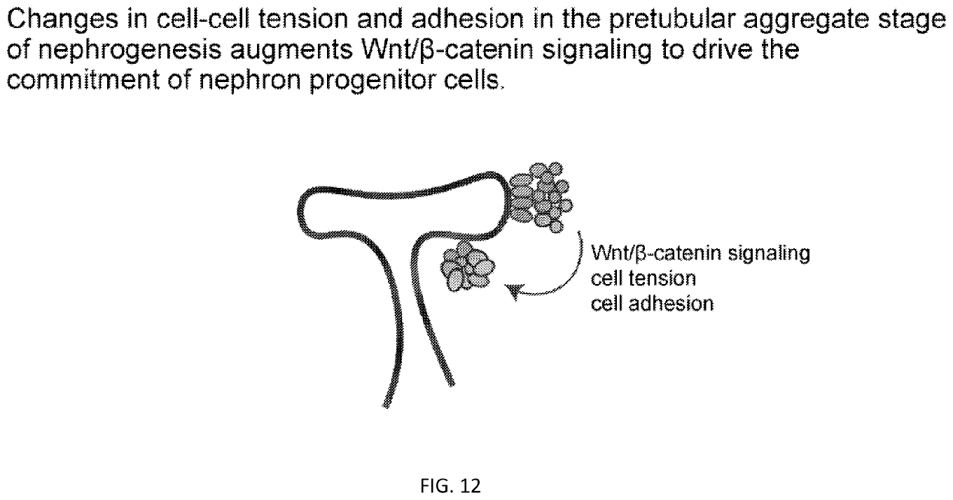

FIG. 13 illustrates an example effect of a cell tension inhibitor on nephron maturation during aggregation.

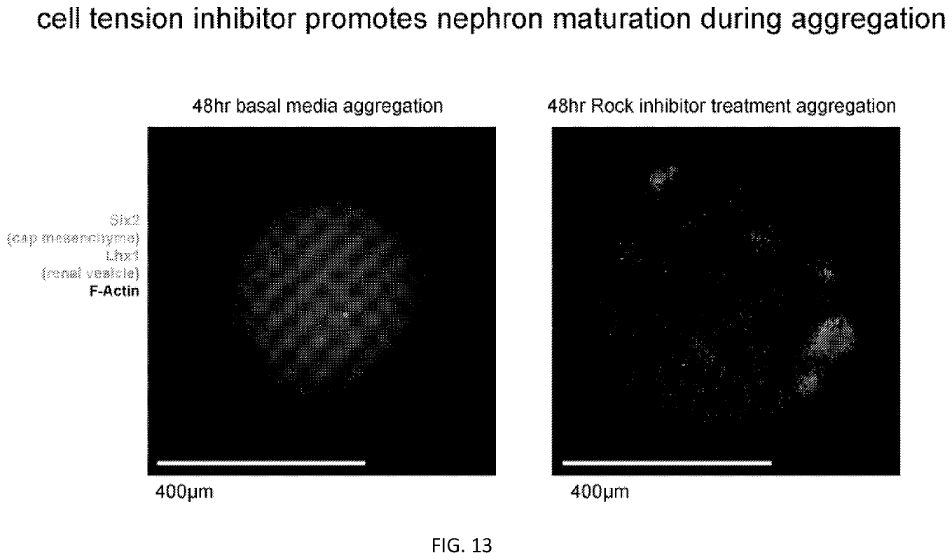

FIG. 14 illustrates an example effect of cell tension inhibitor on nephron maturation during aggregation.

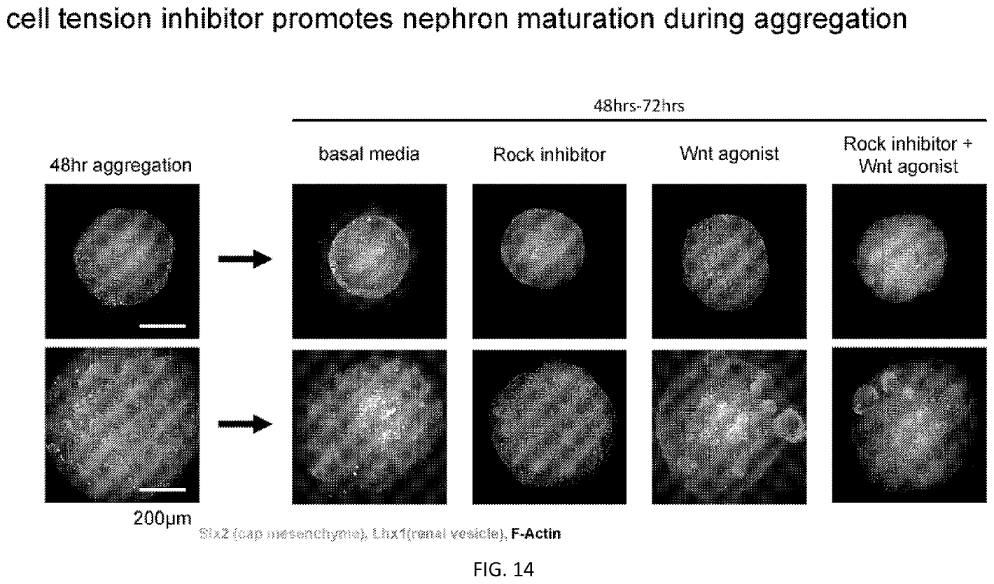

FIG. 15 provides an exemplary, non-limiting description of the principle that attenuation of Wnt/B-catenin signaling is necessary for MET and nephrogenesis progression.

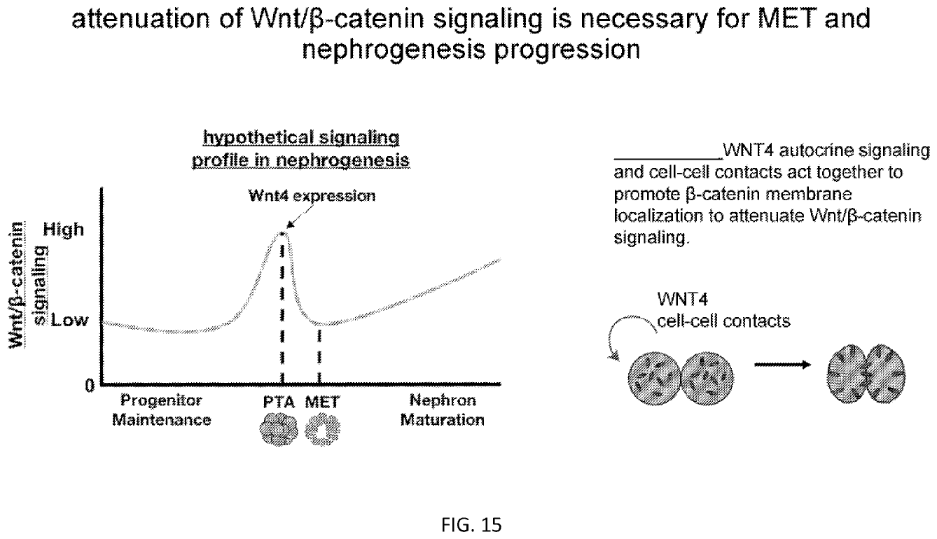

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

The following disclosure is illustrative only and is provided without being bound to any particular theory or embodiment.

### Illustrative Summary

We claim a novel method to increase the nephron yield and control over nephron locations in kidney organoids and kidney replacement tissues. Our method achieves spatiotemporal control over the mechanical microenvironment to engineer favorable environments for nephron formation within kidney organoids. This engineered control can be created using microdevices that impose mechanical stress at defined intervals synchronized with cyclical nephron development in vivo, agonists/antagonists of tension-generating biochemical pathways in whole organoids or cells, optogenetic control over tension in stem cell collectives, and/or cell-cell junction mediated transfer of mechanical information between cells, or similar approaches.

There has been much progress in recent years in the development of kidney organoids and tissue engineered kidneys which would alleviate the need for human donor tissue in treating kidney disease.

However, challenges remain in organoid maturity and higher order organization. These organoid protocols make use of sequential addition of growth factors to recapitulate the signaling pathways activated in kidney development. However, mechanical inputs for differentiation are relatively unexplored. Controlling the mechanical microenvironment in kidney development leands lead to another avenue of control when building kidney tissues, leading to more organized and mature engineered tissues. One particular aspect of kidney development in vivo that is not currently replicated in organoids is the cyclical nature of nephron differentiation.

In mouse and human embryonic kidneys, nephrons form nearby the tips of the developing urinary collecting duct or ureteric bud in waves that synchronize with ureteric tip bifurcation (duplication), whereas in kidney organoids, nephrons form in only one wave. We discovered that nephron production may be dependent on cyclical mechanical cues that track with ureteric tip duplication. In other words, every time ureteric tips divide, there appears to be a pulse of mechanical stress that induces new nephrons in organoid models of this process.

We found that mimicking one cycle of mechanical stress in nephron progenitor organoids derived from human induced pluripotent stem cells significantly increased nephron yield. These data are consistent with an expectation that replicating cycles of mechanical stress will improve nephron yield in kidney organoids, which is crucial for development of new in vitro models of kidney disease, and also for efficient production of syngeneic human tissue for regenerative medicine (replacement kidney tissue).

In addition to changes in the developing nephron mechanical microenvironment, the earliest stage of nephron progenitor differentiation involves a condensation of nephron progenitor cells themselves to form an aggregate. Dysregulated signaling through Yap in nephron progenitors, a mechanosensing protein, has also been noted in mutants with blocked nephron formation and decreased nephron yields. However, changing cell tension and adhesion to drive cell aggregation and condensation with the goal of controlling differentiation has been unexplored. BMP/SMAD and Wnt/beta-catenin signaling through beta-catenin are essential for early nephron differentiation. In addition to its role in Wnt/beta-catenin signaling, beta-catenin also plays a structural role in cell-cell junctions. In fact, it has been shown that force exerted on cell-cell junctions can release structural beta-catenin and promote downstream canonical Wnt/beta-catenin signaling and this phenomenon has been shown to be sufficient to drive germ layer differentiation of stem cells in vitro. This further suggests a link between cell mechanical changes and Wnt/beta-catenin signaling in driving nephron differentiation.

After cap mesenchyme cells are induced to differentiate and form an aggregate, they then undergo a mesenchymal-epithelial transition (MET) to begin to form nephron structures. MET of the aggregate is driven by autocrine signaling through Wnt4, a non-canonical Wnt ligand. Wnt4 signals through Ca(2+)-dependent pathways in NPCs, which is independent of Wnt/beta-catenin signaling. In addition to signaling through Wnt4, for this transition to occur, Wnt/beta-catenin signaling must also be attenuated. In other developmental systems, Wnt4 signaling can direct beta-catenin to the cell membrane in locations of cell-cell contact. In fact, isolated metanephric mesenchyme from mouse embryonic kidney is only competent to epithelialize in vitro when cultured in aggregated 3D contexts in which cell-cell adhesion is high. Cell shape and cell-cell contact lengths are partially controlled through cell cortical tension and cell-cell adhesion. We find that modulating the mechanical microenvironment controls early nephron differentiation as well as at this epithelialization checkpoint through its effects on cell-cell contacts or other mechanical and adhesive cell properties.

We find that the dynamic changes in spatial organization and cell packing that occur in early nephrogenesis are not only the outcomes of differentiation but are also inputs in regulating nephrogenesis progression. We find that these changes in spatial organization and cell packing are driven by a combination of changes in the external mechanical microenvironment and the internal mechanical and adhesion state of nephron progenitor cells. These extrinsic and intrinsic mechanical inputs in nephron progenitor cell differentiation can be controlled through drugs, microdevices and/or optogenetics to spatiotemporally control nephron differentiation within kidney organoids. This leads to larger, more mature, and more structurally true-to-life kidney organoids with higher filtration capacities.

### Illustrative Disclosure

The kidney develops through elaboration of ureteric epithelial tubules (the future urinary collecting ducts), stroma, and nephron progenitors in the cap mesenchyme that surrounds each ureteric tip as they branch. Dynamic interactions between these tissues set nephron numbers for life, impacting the probability of adult disease.

Here we study the geometric and mechanical consequences of tubule tip crowding at the embryonic kidney surface and its effect on nephron formation. We find that kidney curvature reduces and tubule ‘tip domains’ pack more closely over developmental time. These together create a semi-crystalline geometry of tips at the kidney surface and a rigidity transition to more solid-like tissue properties at later developmental stages. New tips overcome mechanical resistance as they branch, expand, and displace close-packed neighbors, after which residual mechanical stress dissipates. This correlates with a changing nephrogenesis rate over the tip ‘life-cycle’. To draw a causal link between the two, we mimic a mechanical transient in human iPSC-derived nephron progenitor organoids and find increased cell commitment to early nephron aggregates. The data suggest that temporal waves of mechanical stress within nephron progenitor populations can constitute a clock that synchronizes nephron formation and ureteric tubule duplication after E15. Ongoing efforts to understand the spatial and temporal regulation of nephron induction will clarify variation in nephron endowment between kidneys and advance engineered kidney tissues for regenerative medicine.

Tissue-building processes during embryonic kidney development set the number of nephrons and urinary collecting tubules in the adult organ, since no further nephrons are added after postnatal day 4 in mice and 36 weeks of gestation in humans (1). The number of nephrons formed in development varies substantially between kidneys and individuals, impacting the likelihood of adult kidney disease (2-4). However, there is limited knowledge on the local and global processes that set nephron numbers. Given that all nephrons are induced by nearby ureteric epithelial tubules, this is at least partially explained by a lack of temporal and spatial consistency in kidney branching morphogenesis. Namely, branching of ureteric tubules is asynchronous and asymmetric (meaning that the number of descendants of tubules at the same branch generation are different) (5,6). Further, while biochemical cues passed between the tips of the ureteric epithelial tree and nephron stem cells in the surrounding cap mesenchyme are well characterized (7-1.1), the time and place at which any given nephron forms cannot be predicted. Gaining a predictive understanding of nephrogenesis is crucial to engineering approaches to kidney replacement technologies and in vitro tissue models, since these must achieve high nephron density and structural connectivity with ureteric tubules to function (12).

During development, the ureteric bud branches into kidney mesenchyme and ureteric tubule tips subsequently engage in Wnt signaling with dynamic clusters of cap mesenchyme cells that proliferate and serve as nephron progenitors (9,13). These signaling interactions and others including in the BMP and Notch pathways induce cap mesenchyme cells to periodically condense and undergo mesenchymal-to-epithelial transition at elbow regions beneath ureteric tips. Nephrons first form as spherical pre-tubular aggregates and later develop into renal vesicles, comma-shaped and S-shaped bodies, simultaneously invading and forming patent lumens with the ureteric tubule and setting the proximodistal axis of the nephron (14,15).

These tissue-building processes are concentrated at the surface of the kidney, with all ureteric tips present at the surface throughout development rather than distributed in deeper tissue layers (16). In recent work, we found that the position of ureteric tips at the kidney surface are partially predictable through a physical model of tip repulsion and crowding (17). Preliminary analysis of tip positions at the surface revealed different crystal-like packing geometries that were at least locally ordered. These observations suggested an analogy to 2D curved crystals of repulsive particles (18-22). Such crystals have several fascinating physical properties that can impact the developmental trajectory of the kidney by setting limits on tip density and therefore nephron number, and by changing the physical microenvironment of tips over their elongation and branching cycles.

The nephron progenitor microenvironment is highly dynamic, unlike many adult stem cell niches in which cells reside in a geometrically fixed microenvironment (for example gut crypts, the basal layer of the skin). This is due to extensive cell motility, proliferation, and division of cap mesenchyme populations among daughter ureteric tubules after each bifurcation event (16,23,24). Despite constant changes in the size of cap mesenchyme populations and proximity of cells to signaling cues produced by ureteric epithelium and stroma (25), the number of nephrons doubles at the same rate that tips do, since the average number of nephrons per tip is fixed at ˜2 over E15-P2 (16). Nephron progenitors can be induced stochastically at exactly the appropriate rate to achieve this (24), but there can also be a clock-like control mechanism operating at the collective cell scale that would tune the induction rate by sensing the time since the last tip duplication, or the local geometry of the cap mesenchyme.

Here we combine the theory of curved crystals, rigidity in close-packed systems, and force inference with analysis of mouse embryonic kidneys and human iPSC-derived kidney organoids to study the interplay between geometry, mechanics, and differentiation in the cap mesenchyme. Our data reveal temporal cycles of mechanical stress within this nephrogenic niche. These cycles may constitute a clock that sets an appropriate frequency of nephron induction by synchronizing nephron formation with the ureteric tip life-cycle.

### Illustrative Results

### Ureteric Tubule Geometry is Partially Determined by Kidney Curvature

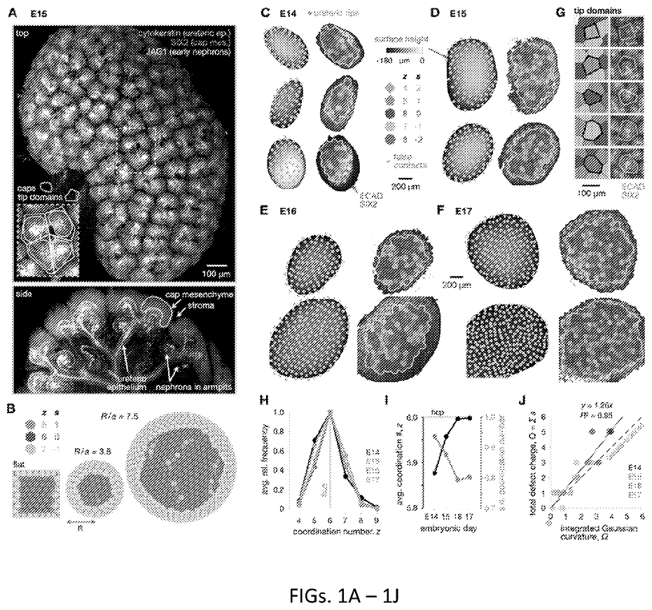

We began by considering the contribution of kidney curvature to the geometric microenvironment of individual ureteric epithelial tips. During branching morphogenesis, ureteric tubules duplicate just beneath the kidney surface. Each tip is surrounded by a swarm of cap mesenchyme cells that serve as nephron progenitors, with each ‘cap’ repelling each other (23,26,27), creating dense arrays of tips separated by thin sheets of stroma (FIG. 1A). We previously noted an analogy for close packing of caps in the physics of repulsive or elastic particles at surfaces (17). Repulsive particles pack most efficiently on flat surfaces in a hexagonal close-packed (triangular lattice) fashion where each particle has six neighbors (i.e. the coordination number z=6) (FIG. 1B). However, wrapping a triangular lattice onto a surface with Gaussian curvature creates an energetic cost that favors the emergence of topological defects called disclinations, in which particles have greater or fewer than six neighbors. Secondly, for curved crystals that are sufficiently large, isolated dislocations are less favorable and are replaced by pairs, clusters, or chains (‘scars’) having ‘excess’ dislocations (Note 1) (18,19,21). We therefore wondered if ureteric tip positions adhere to the same topological requirements that set defect number and organization in curved crystals.

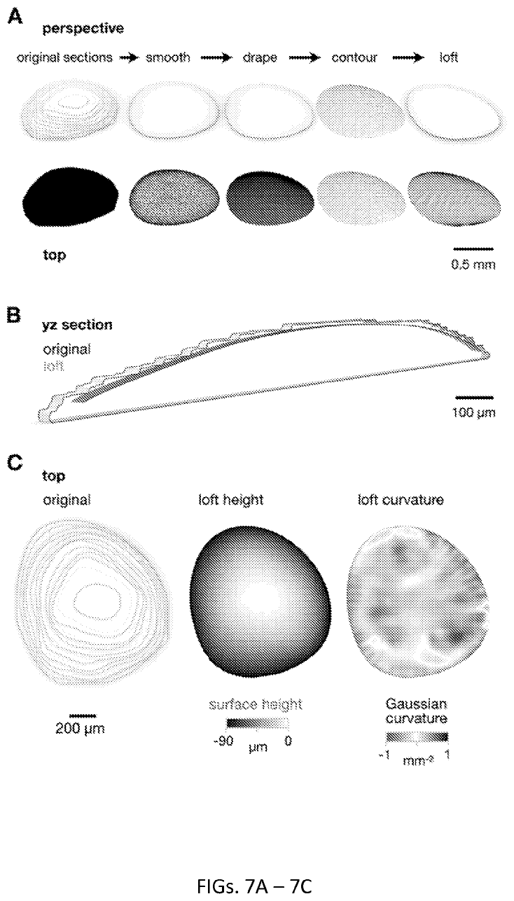

We began by annotating ureteric tip positions on kidneys over embryonic days E14-E17 and extracting the predicted lattice boundaries of each tip ‘domain’ (all ureteric epithelium, cap mesenchyme, and stroma closer to a given tip than to a neighboring one) using a Voronoi tessellation approach. We then separately reconstructed their surfaces from 3D confocal image stacks to compute their curvature maps (FIG. 7). Tip positions and Voronoi diagrams describing tip domains are shown in FIG. 1C-F. We also colored tip domains according to the number of neighbors they contact (the coordination number z), which is related to their ‘topological charge’ s=6−z (FIG. 1G). Tip coordination numbers varied between 4 and 8, and tip patterns qualitatively transitioned from a disordered state at E14-E15 to more visually apparent local order at E16-E17 (see also (17)). Voronoi analysis successfully predicted the position and boundaries of tip domains (FIG. 1G), suggesting that their size and shape are restricted by the presence of neighboring domains. The median coordination number of tips was z=6, with a bias toward lower mean coordination number at earlier developmental times. This is consistent with tip patterns adopting energetically favorable pentagon disclinations (i.e. tips with z=5 neighbors instead of 6) in younger kidneys with higher curvature (FIG. 1H,I). At later times, the coordination number distribution narrowed and approached a mean of 6, consistent with predictions for particle packings on flat surfaces (FIG. 1I). The theory of curved crystals suggests that the amount of bias toward pentagon dislocations will increase linearly with curvature (Note 2). Indeed, we find substantial agreement with the prediction of a bias toward pentagonal tip domains for younger kidneys with greater curvature per confocal region of interest (FIG. 1J). While variation in tip domain size and shape appears to contribute to the majority of excess dislocations in the kidney case, we also see evidence of grain boundary scars and clusters of defects of alternating charge (FIG. 1C-F). These data show that tip packing geometry at the kidney surface is partially determined by topological limitations imposed by kidney curvature.

### Crowding of Ureteric Tip Domains Creates Crystal-Like Packing Patterns and a Mechanical Rigidity Transition

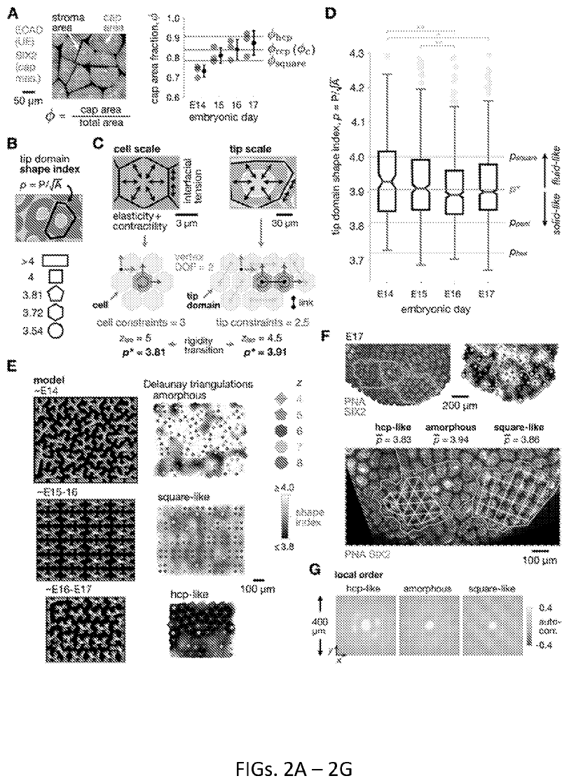

Beyond the contribution of curvature to the geometric microenvironment of ureteric tip domains, we sought to understand the impact of crowding among neighboring caps, each bordered by intervening layers of stroma. During early branching morphogenesis, caps and stroma are both fluid-like on a developmental timescale (23,28) (Note 3). However, the stroma thins over E14-E18, causing caps to pack closer together, conform in shape because of the confinement imposed by neighboring caps, and locally align with each other (17). Similar geometric features are observed in confined packings of soft elastic spheres (29) or closely packed droplets in dense two-phase emulsions (30-32). Since the degree of crowding can create abrupt changes in the geometric and mechanical properties of these systems, we wondered if similar effects could occur during cap packing. One such property is a so-called density-dependent rigidity (jamming) transition that occurs when droplets crowd together beyond a critical volume fraction ϕ_{c} and transition from zero to some finite yield stress (i.e. from fluid-like to solid-like behavior) (33,34). For kidney caps, we determined ϕ on a 2D basis as the ratio of cap area to total area. This area fraction ϕ exceeded those for 2D body-centered (square) packing of circles and for random close packing (which defines ϕ_{c} (33,35,36)), even approaching that for 2D hexagonal close packing (hcp) after ˜E15 (FIG. 2A). This predicts a jamming transition, such that even though caps and stroma are both fluid-like, the surface as a whole is predicted to be solid-like and therefore capable of imposing mechanical stress on newly formed caps during tip duplication after ˜E15.

Although ϕ predicts density-dependent jamming of caps, the underlying theory reduces potentially important parameters to a passive interfacial tension (33,37). These parameters include collective cell elasticity and active contractility (embryonic kidney cortex explants shrink in the hours after cutting (17)), and extra contributions to interfacial tension at cap-stroma boundaries (tissue layers are adhered through cell-cell junctions and/or cell-extracellular matrix interfaces). We therefore made some further predictions using a density-independent rigidity theory created for the high packing fraction regime that does consider these parameters. This theory considers jamming in 2D cell monolayers using a cell vertex model (38-40). We instead applied it at the higher-level organizational scale of tip domains, since the underlying mechanical energy terms are equally applicable to tip domains as to cells (see Methods). The vertex model as applied to cells predicts a transition to rigidity when the median of a geometric parameter of cell boundaries called the shape index p (FIG. 2B) drops below a critical shape index p* (38,39). When p>p*, there is no energy barrier to transitions between different packing configurations, conferring fluid-like mechanics. However, when p<p* the configuration of cells has both bulk and shear stiffnesses, conferring solid-like mechanics (40).

One explanation for a fluid-like to solid-like transition upon reducing median shape index comes from rigidity theory, in which a mechanical assembly is rigid when the number of degrees of freedom of nodes matches the number of constraints (41,42). This predicts rigidity for a polygonal tiling of cells when p<p* =3.81 (above z=5) (38), and when p<p*=3.91 (above z=4.5) for tip domains (FIG. 2C, Note 4). We measured the shape index of tip domains over E14-E17, finding that the median shape index significantly decreased, dropping below 3.91 after E15 (FIG. 2D). Despite a relatively small effect size, the change in shape index here is in a similar range to that previously associated with a rigidity transition in airway epithelial cells (39). This result suggested that the mechanical microenvironment of tips can become stiffer and less viscous after E15, consistent with the time at which crystal-like locally ordered regions began to appear in FIG. 1.

To draw a connection between shape index and our previous geometric model of tubule family packing, we produced heat maps of tip domain shape index for three characteristic regimes of tip packing that we identified in previous work (17). These three regimes, which at the tip level can be qualitatively referred to as amorphous, square-like, and hcp-like respectively, mirror the reduction in tip domain shape index from E14-E17 (FIG. 2E). In reality, tip packing does not perfectly adhere to any one regime at a snapshot in time (FIG. 2F), probably because asynchronous tip duplication creates heterogeneity (polydispersity) in the relative size and shape of tip domains. This means that our previous model predicts the stage at which a given packing phase has the potential to exist, but does not account for a mixture of phases that arises due to tip domain polydispersity. For example, at E17 we observe short-range regions that exhibit amorphous, square-like, or hcp-like packing geometry that persists spatially for 2-3 tip spacings translationally, as revealed by spatial autocorrelation analysis showing four-fold and six-fold rotational symmetry for square-like and hcp-like regions respectively (FIG. 2G). Overall, these data show that tip packing at the kidney surface is semi-crystalline, with tip domains having shape indices consistent with an increased prevalence of square packed and hcp regions at later developmental times.

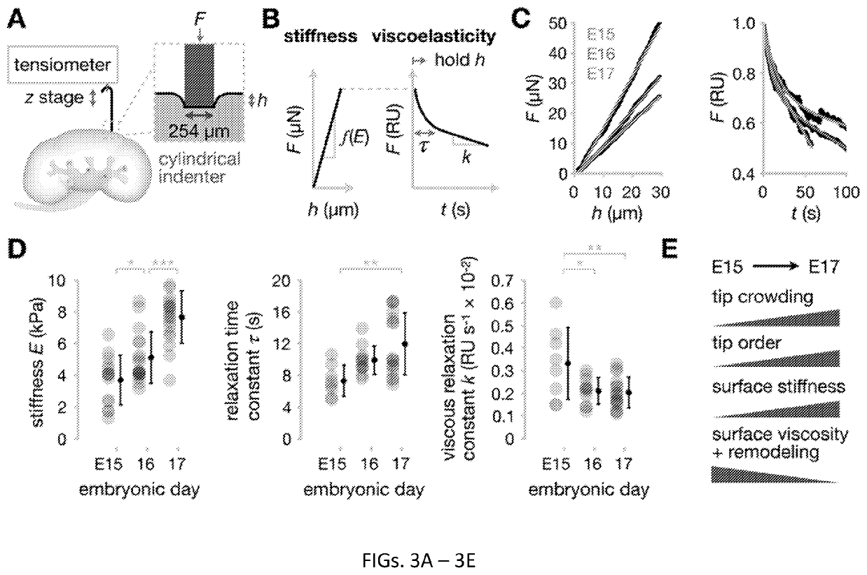

We next explored whether predictions from rigidity theory and the increase in crystalline order of tubule tips over time would correlate with tissue mechanical properties on the length-scale at which tip domains interact. Taking advantage of the location of tip domains at the kidney surface, we used surface microindentation to quantify surface elastic modulus (stiffness) and viscoelasticity over E15-E17 using a 254 μm cylindrical indenter (equivalent to the width of ˜3 tip domains, FIG. 3A, see Methods) (43). We measured the applied force recorded during indentation of the kidney surface by ˜30 μm, and paused indentation to capture the time dependence of force during subsequent tissue relaxation (FIG. 3B). These measurements revealed a significant increase in kidney surface stiffness local to tip domains over E15-17 (FIG. 3C,D). Secondly, they showed an increase in the time-scale of force relaxation over E15-E17, perhaps due to a slowing in passive remodeling of cell collectives and extracellular matrix (43) (FIG. 3C,D). Thirdly, the measurements showed a decrease in viscous relaxation over E15-E17, perhaps caused by slowing of active tissue remodeling. Together, these data reveal marked increases in stiffness and decreases in passive and active tissue remodeling over E15-E17, the same time period over which our tip domain shape index analysis predicted a fluid-like to solid-like transition in tissue rigidity (FIG. 2D, FIG. 3E).

An increase in kidney surface stiffness and reduction in viscosity would increase the mechanical resistance to new tip domains forming, expanding, and displacing close-packed neighbors. This would imply that tip domains see cyclical swings in their mechanical microenvironment on a similar timescale to tip duplication events. The local packing state can even influence the balance of tip duplication and nephrogenesis events, either because of geometric changes in morphogen and growth-factor presentation to nephron progenitors, or because of mechanical influences on cell signaling. Mechanical modulation of progenitor differentiation can occur via intersection with Wnt/β-catenin signaling, for example in mesoderm specification of hESCs (44) and in morphogenesis of the avian feather primordium (45). Mechanical modulation can also occur via intersection with TGFβ/pSMAD signaling, for example in translating anisotropic stress in the condensing digit mesenchyme into expression of digit organizing center genes (46). In the kidney cap mesenchyme, both of these potentially mechanosensitive Wnt/β-catenin and TGFβ/pSMAD signaling axes (along with YAP (47)) are required for nephrogenesis (see Note 5 for detail). However, the potential influence of a mechanical context in tip domains that fluctuates on the timescale of nephrogenesis has not been determined either in vivo or using stem cell models.

### Nephrogenesis Rate Varies Over Tip Domain Life-Cycle

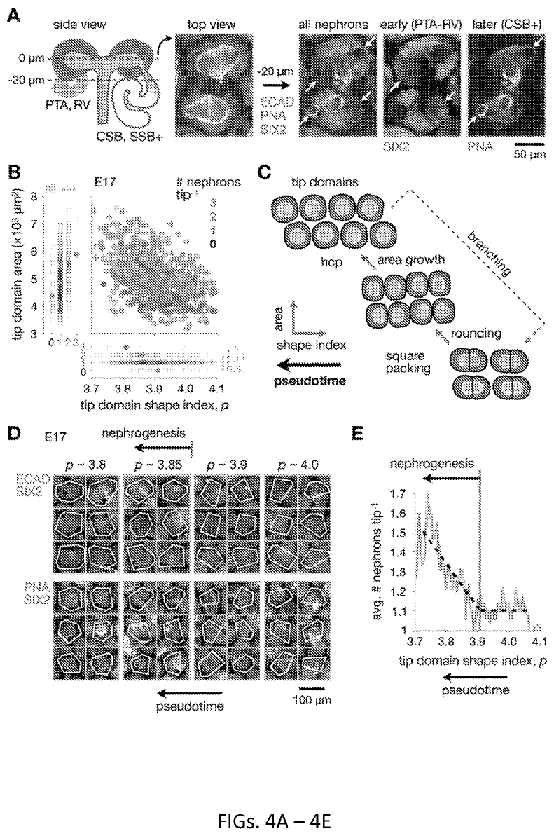

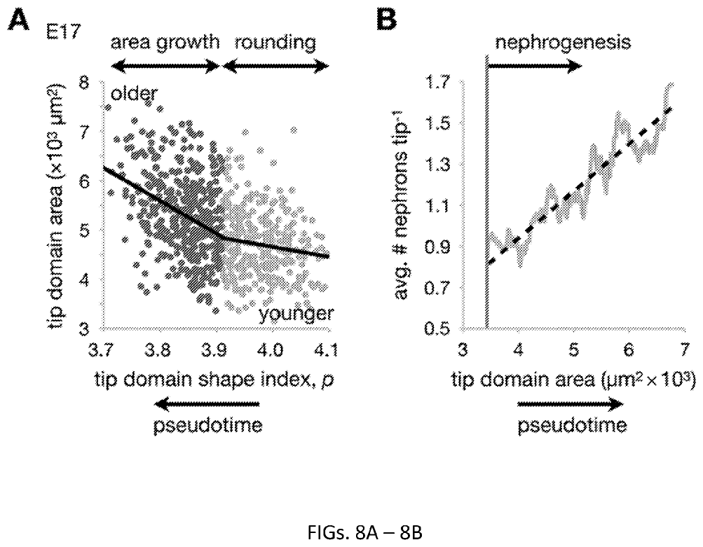

If periodic swings in the geometric or mechanical microenvironment of tip domains affect nephrogenesis, one would expect to see periodic changes in nephrogenesis rate over the life-cycle of tip domains. However, it is not currently possible to live-image tip geometry and nephron formation in kidney explants while retaining their in vivo 3D architecture (48), so we instead attempted to extract correlative information from fixed explants. We focused on E17 kidneys, reasoning that periodic increases in local mechanical stress due to tip duplication would be amplified due to their higher surface stiffness and lower viscosity relative to earlier stages. Nephrons can be scored from immunofluorescence stacks by combining annotations of Six2+ spheroids beneath ureteric tips (capturing pre-tubular aggregate and renal vesicle stages) with annotations of sites where connecting tubules from more mature nephrons connect to tips (capturing comma shaped body, S-shaped body, and later stages), FIG. 4A. First, we found a negative correlation between tip domain area and shape index, which appears to reflect the ‘life-cycle’ of tips. Specifically, recently divided tips have lower area and higher shape index, while older tips are larger with lower shape index (FIG. 4B). The inference of tip age is revealed when we overlay the number of nephrons associated with each tip, namely that nephron number increases as tips grow in area and become more circular. When tips divide, their nephrons are divided among daughter tips (16), and the cycle repeats (FIG. 4C). These data imply that the tip domain shape index can be used as a “pseudotime” dimension that corrects for the lack of synchronization of tip duplication events. In other words, tips in different locations can be aligned with respect to their progress through morphogenesis relative to their last duplication event (FIG. 4D). This enabled us to plot a rolling average of nephron number per tip against shape index. If nephron progenitors commit to early nephron aggregates stochastically (24) and at a fixed rate, we would not expect the nephrogenesis rate to depend on tip life-cycle. However, nephrogenesis appears to switch on as the shape index of tip domains drops below an intermediate value of p˜3.91 and the area of tip domains begins to increase (FIG. 4E, FIG. 8). This means that nephrogenesis pauses as new tips push outward against neighboring tip domains and begin to round up, and resumes as tip domains then grow in area.

### Newly Branching Tip Domains Experience Mechanical Stress That Dissipates Over Their Life-Cycle

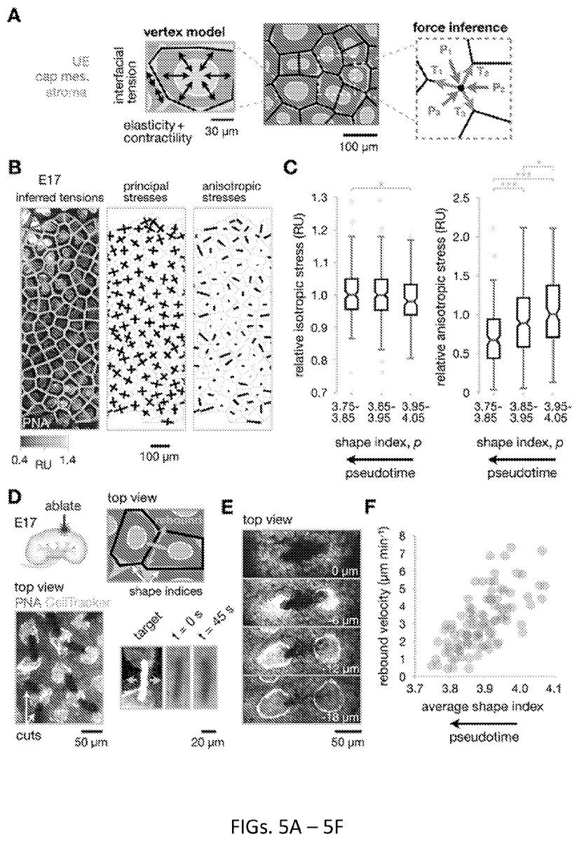

After finding that nephrogenesis rate varies periodically with tip life-cycle, we wondered if mechanical stress local to tips would also depend on their life-cycle. To assess this, we drew on force inference methods developed from cell-scale vertex modeling to investigate mechanical heterogeneity in groups of ureteric tip domains (49-51). Force inference has been successfully validated by laser ablation experiments across several model organisms, tissue types, and length-scales (from several-cell to highly multicellular tissues) (51). We again reasoned that force inference could be extended to the analysis of tissue-level tip domain populations because both cells and tip domains can be described by equivalent mechanical energy terms in the underlying cell vertex model (FIG. 5A, Methods). We therefore performed force inference on Voronoi diagrams produced from E17 kidney surface projections to recover the inferred stress tensor, which yields major and minor principal stress axes and relative magnitudes that then give relative isotropic and anisotropic (deviatoric) stresses (FIG. 5B). We plotted these components against the shape index of tip domains to show that as tips age relative to their last duplication event (i.e. as shape index decreases), they see a marginal increase in inferred isotropic stress, and a ˜30% decrease in anisotropic stress (FIG. 5C). This indeed suggests a cyclical mechanical environment synchronized with the tip life-cycle, and in particular, that anisotropic stress immediately precedes the nephrogenic phase and then falls most substantially as tip domain shape index drops below p˜3.91 (FIG. 4E).

We next sought to validate inferred stresses local to tip domains experimentally. We used laser microdissection of E17 kidney explants to create slot-like ablations spanning neighboring cap mesenchyme populations and orthogonal to the stromal boundary between pairs of ureteric tips (FIG. 5D). Laser ablation is thought to most closely reflect anisotropic stress at cutting sites (50), such that the rebound (opening) velocity of cut edges is proportional to local tension (51). Ablation successfully induced rebound at cut sites (FIG. 5D), and cuts penetrated ˜40 μm in depth, sufficient to sever the mesenchyme and stroma between tips over their full z extent (FIG. 5E). Validating the force inference data, the rebound velocity of cuts increased with the average shape index of tip domains adjacent to each cut (FIG. 5F). These data show that anisotropic stress local to the cap mesenchyme is highest for newly duplicated tips with higher shape index and lowest for older tips with lower shape index. This appears to validate our earlier notion that new tips overcome mechanical resistance as they branch, expand, and displace close-packed neighbors, after which mechanical stress dissipates.

After finding that nephrogenesis rate and tip domain mechanics vary periodically with tip life-cycle, we wondered if the two were causally connected. Although SIX2+ nephron progenitor induction via TGFβ/pSMAD and Wnt/β-catenin signaling axes is crucial for nephrogenesis, perturbations apparently unrelated to ligand secretion or spatial distribution appear to affect it (see Note S6 for detail). These include perturbations to kidney explant culture geometry and mechanics as well as to Rho/ROCK and non-muscle myosin II, which are important for cell tension and perception of the mechanical microenvironment (52,53). Such disparate observations have not previously been interpreted in the light of mechanics of the nephrogenic niche, motivating further investigation.

### Mimicking Transient Cell-Collective Stress During Tip Domain Branching Increases Nephrogenesis in Kidney Organoids

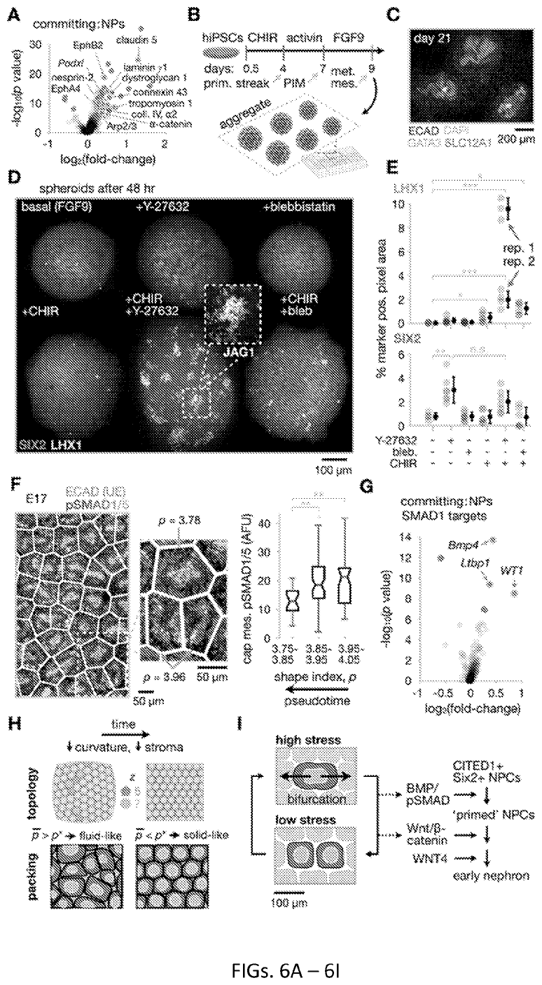

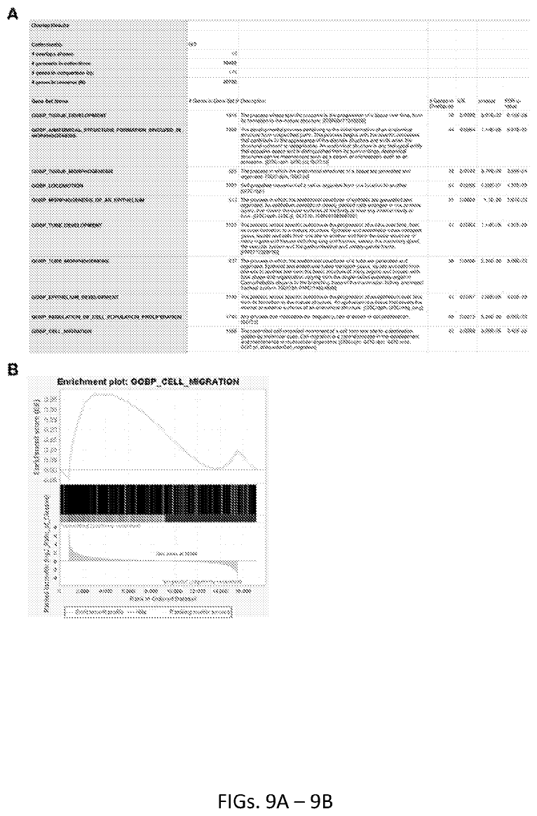

Cytoskeletal tension and adhesion properties of cells often reflect biophysical properties of the tissue microenvironment, which can direct cell decision-making (44,54-57). We wondered then if nephron progenitors perceive stiffness and tension changes in the tip domain microenvironment over the tip life-cycle. We began by analyzing differential gene expression in a recently published scRNA-seq analysis of nephron progenitor and committing cell sub-populations of E15.5 mouse kidneys (24). Lawlor et al. confirmed differential expression of marker genes previously ascribed to these populations, including Cited1 and Six2 for nephron progenitors and Wnt4, Pax8, Lhx1, and Jag1 for committing cells (24). The top 10 Gene Ontology (GO) terms (58) associated with genes significantly upregulated in committing cells compared to progenitor cells included cell locomotion and cell migration, processes in which cell cytoskeleton, adhesion and mechanical properties change (59) (FIG. 9). We therefore examined differential expression of genes combined from the cell migration GO term and pathways associated with cytoskeletal remodeling from the PathCards database (60). We found significant increases in transcripts involved in cytoskeletal integrity and remodeling (nesprin-2, tropomyosin, Arp2/3), cell-cell junctions and adhesion (Claudin 5, Podxl, connexin 43, α-catenin), and cell-extracellular matrix adhesion (dystroglycan 1, collagen IV, laminin) in committing cells prior to significant differential E-cadherin expression, lending evidence of a change in their mechanical state prior to early nephron epithelialization (FIG. 6A).

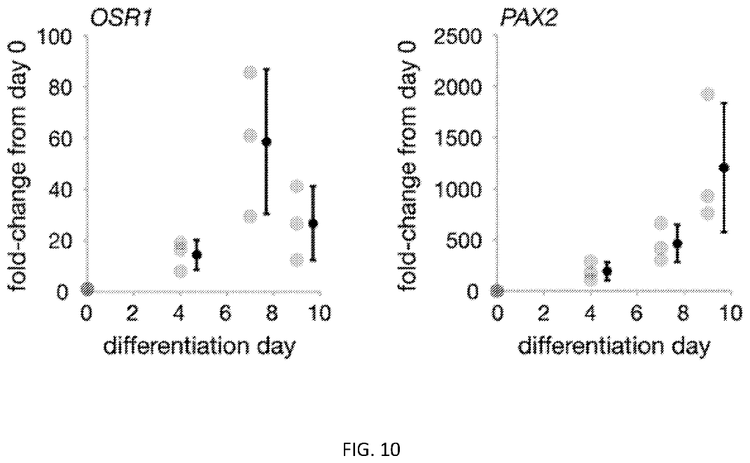

We next turned to a bottom-up approach using human iPSC-derived nephron progenitor organoids to assess the effects of mechanical microenvironment changes on nephron differentiation (FIG. 6B). When differentiated, these roughly mimic the progression in transcriptional states and marker expression profiles of mouse nephron progenitors in vivo, despite some species-specific differences (61-65). To test perturbations to cell collective tension during nephron progenitor commitment, we first differentiated PSCs through late primitive streak and posterior intermediate mesoderm (PIM) lineages to metanephric mesenchyme according to established protocols (66), confirming by qPCR (FIG. 10). We also validated that induced metanephric mesenchyme cells go on to form later nephron structures including ECAD+tubules containing GATA3+ distal nephron cells and Na⁺/K⁻/2Cl⁻ cotransporter SLC12A1+ loop of Henle cells after 21 days in culture (FIG. 6C). Day 9 induced metanephric mesenchyme cells were dissociated and plated into low-attachment round-bottom plates where they formed spheroids that we assayed for early nephrogenesis (FIG. 6D). In similar assays, a CHIR pulse is typically applied after re-aggregation of cells to mimic Wnt9b-driven β-catenin signaling during nephron progenitor commitment in vivo (61,62,67,68). We observed that CHIR also induced cell release from their substrate and compaction in day 9 iPSC-derived metanephric mesenchyme that appeared to occur due to collective cell contractility rather than motility. We took these cells as a model for Wnt9b-stimulated nephron progenitors in the high anisotropic stress state shortly after tip duplication.

We then decided to mimic the reduction in anisotropic stress that occurs over the tip life-cycle in vivo by adding the ROCK inhibitor Y-27632 or the non-muscle myosin II inhibitor blebbistatin to day 9 metanephric mesenchyme spheroids for 48 hr. These temporal parameters were optimized to capture early transitions of cells through the SIX2+ state and to accumulate differentiation events sparsely, enabling quantification. Spheroids were analyzed by immunofluorescence for SIX2 and the pre-tubular aggregate/renal vesicle (early nephron) marker LHX1 (FIG. 6E). We found that Y-27632 increased cell conversion to and/or self-renewal of SIX2+ progenitors, but did not by itself induce LHX1+ cells. CHIR increased differentiation to the LHX1+ state as previously described (61), and combining CHIR with Y-27632 or blebbistatin had a synergistic effect. These LHX1+ cells clustered and began to express the later medial nephron marker Jag1, indicating appropriate progression of differentiation after perturbation (FIG. 6D). These data suggest that Wnt-driven nephron progenitor commitment may be licensed by a collective cell microenvironment with lower mechanical tension/anisotropic stress.

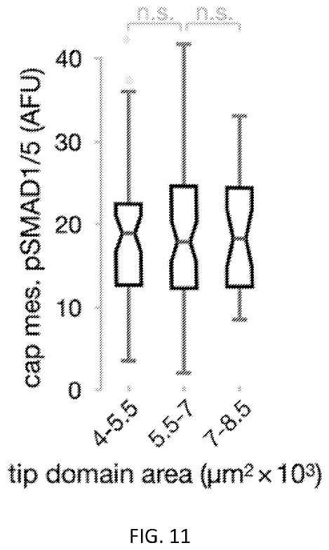

We next considered candidate signaling pathways involved in nephron progenitor commitment that are known to be mechanosensitive in other contexts. We focused our attention on quantifying pSMAD1/5 by whole-mount confocal immunofluorescence due to its proposed mechanosensitive properties and role in BMP7-mediated transition of CITED1+SIX2+ nephron progenitors to a CITED1−SIX2+ state ‘primed’ for further induction via Wnt/β-catenin (46.69). We observed that pSMAD1/5 appeared to be enriched in cap mesenchyme cells within recently branched tip domains since mean pSMAD1/5 levels in caps positively correlated with the shape index of their tip domains, but not their area (FIG. 6F, FIG. 11). These data showed that pSMAD1/5 in nephron progenitors is sensitive to changes in shape of tip domains, perhaps due to differences in the geometric or mechanical microenvironment. Analysis of published scRNA-seq data confirmed that genes having a SMAD1 binding motif around their transcriptional start sites including BMP4 and WT1 are upregulated in committing cells compared to progenitors (FIG. 6G) (24). BMP4 is redundant with BMP7 in priming nephron progenitors (69), perhaps indicating a positive feedback mechanism for priming. While also negatively regulating SMAD1/5 itself (70), WT1 activates the expression of Wnt4 in kidney mesenchyme and is essential for early nephron epithelialization (71), indicating that the role of pSMAD1/5 in nephron progenitor priming (69) can synergize with later Wnt/B-catenin induction events. These data show that SMAD signaling relevant to progenitor induction is spatiotemporally correlated with tip life-cycle.

### Discussion

Gaining a fundamental understanding of tissue-wide coordination between ureteric epithelial and nephron morphogenesis provides control strategies for addressing variability in nephron endowment between kidneys (72-74). However, nephron number is set by complex signaling interactions within and between cell populations local to ureteric tips. Perturbing levels of key factors including Six2 (75) and Tsc1 (72) can have paradoxical effects on nephrogenesis, and the same pathway can regulate both nephron progenitor renewal and differentiation at different levels or in closely related cell states (Wnt/β-catenin (76,77)) or through different downstream effectors (BMP/MAPK vs. BMP/SMAD (73)). Kidney organoids constitute both a promising platform to clarify the fundamentals of nephrogenesis and a vehicle toward augmentation of adult kidney function. However, nephrons form in a single synchronous wave in iPSC-derived kidney organoids rather than periodically (in the reference frame of individual ureteric tips) (12). Replicating this in organoids is a significant opportunity since successive rounds of nephrogenesis is crucial to achieving high nephron density in vivo.

Despite evidence of tight control over nephron progenitor differentiation at the signaling level, an overarching clock that explains the consistent number ratio of nephrons to ureteric tips seen over E15-P2 (16) has been elusive. Progenitors stochastically enter and exit concave ureteric tubule ‘armpit’ regions that might concentrate secreted inductive factors, but no such shape-dependence has been found (24,76). We decided instead to characterize the geometric reference frame of tip domains over an inferred life-cycle to uncover other possibilities. Our results suggest that topological requirements imposed by decreasing curvature of the kidney and closer packing of tip domains forces them into semi-crystalline organization after ˜E15 (FIG. 6H). This coincides with an increase in solid-like properties at the kidney surface and mechanical stress local to tip domains that correlate with their life-cycle (FIG. 6I). Physical resistance to tip duplication can mean that each tip domain experiences temporal waves in mechanical stress that we hypothesize can synchronize nephron formation with ureteric epithelial branching.

The compositional and dynamic complexity of kidney morphogenesis drove us to reductionist perturbations of cell collective tension in nephron progenitor spheroids, which model early nephrogenesis (61,62,69). Perturbing collective cell contractility to model the lower stress nephrogenic microenvironment later in the tip domain life-cycle increased nephrogenesis in this in vitro system. Although we do not directly identify the mechanotransduction pathways involved, our organoid and explant data suggest further investigation of mechanical modulation of TGFβ/pSMAD and Wnt9b/β-catenin pathways. Our data fit a hypothesis previously applied to the cessation of nephrogenesis, where increasing BMP7/SMAD signaling at the expense of BMP7/MAPK is predicted to increase differentiation of NPCs by sensitizing them to Wnt/B-catenin mediated differentiation at the expense of self-renewal as development progresses (69,73,78). However, we apply a similar logic on the timescale of tip duplication (FIG. 6I). In this narrative, a pulse of mechanical stress at the beginning of each tip duplication cycle increases BMP7/pSMAD activity in analogy to its role in digit formation (46). This can prime an appropriate fraction of nephron progenitors for commitment during each tip duplication cycle. However, other narratives are partially consistent with the data. For example, a high tip domain shape index shortly after branching can increase contact surface area of FOXD1+FAT4+ stroma with nephron progenitors, which are known to promote their differentiation (79-81). The two narratives are not mutually exclusive, since for example the effect of Fat4 intersects later during commitment of primed cells via Wnt9b/B-catenin.

Distinguishing between these hypotheses would benefit from additional mechanical and nephrogenic signaling perturbations in organoids, mouse cap mesenchyme cultures (82), and kidney explants. The latter requires innovation of new live culture and imaging techniques that preserve kidney shape in 3D, especially to study branching dynamics after ˜E15. An atlas of nephron progenitor cell biophysical properties such as traction and adhesion along their developmental trajectory would also aid in aligning differentiation decisions with biochemical and mechanical signaling inputs. Altogether, a broader understanding of connections between kidney geometry, mechanics, and niche signaling provides for engineering control over the time and place of nephron formation in regenerative medicine applications.
Note 1. Repulsive particles pack most efficiently on flat surfaces in a hexagonal close-packed (triangular lattice) fashion where each particle has six neighbors (i.e. the coordination number z=6) (FIG. 1B). However, this packing pattern cannot be maintained on a curved surface, since wrapping a triangular lattice onto a surface with non-zero Gaussian curvature creates strain (83). This strain creates an energetic cost that favors the emergence of topological defects called disclinations, in which some particles have greater or fewer than six neighbors. For example, the strain introduced into a hexagonal lattice mapped onto a spherical surface can be relieved by introducing 12 pentagons (known as topological defects of ‘charge’ s=+1), such as in the familiar pattern of pentagons and hexagons making up a soccer ball. For curved crystals that are sufficiently large (R/a>5 for spheres, where R is sphere radius and a is mean particle spacing), the energy cost of isolated pentagon (s=+1) defects becomes too large (19,84). The resulting strain is therefore relieved through the spontaneous appearance of ‘excess’ dislocations as pairs, clusters, or chains of alternating charge (i.e. particles with coordination number 5-7-5-7- . . . -5) called ‘grain boundary scars’ each with a net charge of +1 that effectively distribute the elastic energy of the defect over a larger area (18,19,21).
Note 2. The theory of curved crystals suggests that the amount of bias toward pentagon dislocations will increase linearly with curvature. This curvature dependence of tip geometry can be summarized by plotting total disclination defect charge Q for individual kidney fields of view against the surface integral of their Gaussian curvature (in units of disclinations), Q. The Gauss-Bonnet theorem suggests that the overall topological charge will increase linearly here to “screen” curvature-induced stress (18,20).
Note 3. Live imaging of cap mesenchyme cells in mouse kidney explants at an equivalent developmental time of ˜E14 has shown their mean speed to be approximately 3.5×10⁻³ μm s⁻¹ (12.6 μm hr⁻¹), compared to a relative divergence speed of sister ureteric epithelial tip domains of ˜2-5 μm hr⁻¹ (23). Therefore, during early branching morphogenesis, cap reorganization is sufficient to consider it as a fluid-like compartment on the timescale of tip domain duplication. Less is known about stromal cell dynamics, but endothelial cell induction from FOXD1+ stromal cells, recruitment, and reorganization into developing vascular plexuses at the border of tip domains is synchronized with their duplication (28), suggesting that cell dynamics in the stroma are also sufficient to consider it as a fluid-like compartment.
Note 4. For a polygonal tiling of cells, there are 2 degrees of freedom for each vertex (motion in x and y) and 3 constraints per cell (that would enforce force balances in x and y, and torque balance). Equating degrees of freedom and constraints and using Euler's formula then predicts rigidity for p<p*=3.81 (above z=5, FIG. 2C) (38). In the case of ureteric tip domains, there is one fewer translational constraint per pair of tips needed for rigidity, since each tip is physically linked to a sister tip. This instead predicts rigidity for p<p*=3.91 (above z=4.5) (FIG. 2C). See Methods for further detail.
Note 5. In the kidney cap mesenchyme, both of the potentially mechanosensitive Wnt/β-catenin and TGFβ/pSMAD signaling axes (along with YAP (47)) are required for nephrogenesis. Firstly, pSMAD signaling downstream of BMP7 is necessary to ‘prime’ nephron progenitors for induction by Wnt9b/β-catenin to a WNT4+ state capable of contributing to an early nephron aggregate (69). However, not all WNT4+ cells commit and can return to the progenitor pool, suggesting that an extra mechanical or other unknown cue may be required (24). Secondly, after its initial activation, β-catenin signaling must be downregulated for fully epithelialized renal vesicles to emerge from WNT4+ aggregates (67,68). Indeed, tuning microenvironmental factors such as cell density and temporal dynamics of the β-catenin transient using the GSK3β inhibitor CHIR are crucial to nephrogenesis in human iPSC-derived kidney organoid differentiation (61,62,66).
Note 6. Other perturbations apparently unrelated to ligand secretion or spatial distribution appear to affect nephrogenesis. For example, nephrogenesis is significantly increased during embryonic kidney explant culture under surface tension at an air-media interface, rather than suspended just below the media surface or in hanging drops (85). Nephron formation in hemispherical bioprinted organoids is biased toward high-curvature edges (86), which are known to amplify mechanical strain (44). Nephron progenitor differentiation is dependent on YAP (47), an integrator of mechanical cues affecting a range of differentiation events (87,88). Stromal Fat4 enhances nuclear export of YAP in nephron progenitors, tipping their perception of Wnt/β-catenin signaling toward differentiation (79). Finally, negative regulation of Rho GTPase, Rho-associated protein kinase (ROCK), and non-muscle myosin IIA/B isoforms (Myh9/Myh10) lead to increases in nephron formation (low dose H1152, a ROCK inhibitor) or decreases in nephron formation (high dose H1152; mesenchymal Myh9/Myh10 deletions) (89,90). Both Rho/ROCK and non-muscle myosin II are relevant to cell tension and perception of the mechanical microenvironment (52,53).

### Methods

Animal experiments. Mouse protocols followed NIH guidelines and were approved by the Institutional Animal Care and Use Committee of the University of Pennsylvania. E14-E17 embryos were collected from timed pregnant CD-1 mice (Charles River) and stages confirmed by limb anatomy as previously described (91). Embryonic kidneys were dissected in chilled Dulbecco's phosphate buffered saline (DPBS, MT21-31-CV, Corning) (92).

Kidney immunofluorescence imaging. Immunofluorescence staining and imaging was performed as previously described (17), using protocols adapted from Combes et al. and O'Brien et al. (93,94). Briefly, dissected kidneys were fixed in ice cold 4% paraformaldehyde in DPBS for 20 min, washed three times for 5 min per wash in ice cold DPBS, blocked for 2 hr at room temperature in PBSTX (DPBS+0.1% Triton X-100) containing 5% donkey serum (D9663, Sigma), incubated in primary and then secondary antibodies in blocking buffer for at least 48 hr at 4° C., alternating with 3 washes in PBSTX totaling 12-24 hours. The minimum duration of primary and secondary incubations and washes depended on the age of the kidney, as previously described (93).

Primary antibodies and dilutions included rabbit anti-Six2 (1:600, 11562-1-AP, Proteintech, RRID: AB_2189084), mouse anti-E-cadherin (clone 34, 1:200, 610404, BD Biosciences, RRID: AB_397787), mouse anti-calbindin D-28K (clone CB-955, 1:100, C9849, Sigma, RRID: AB_476894) mouse anti-pan-cytokeratin (clone 11, 1:200, C2931, Sigma, RRID: AB_258824), goat anti-jagged 1 (1:150, AF599, R&D Systems, RRID: AB_2128257). Secondary antibodies (all raised in donkey) were used at 1:300 dilution and include anti-rabbit AlexaFluor 647 (A31573, ThermoFisher, RRID: AB_2536183), anti-rabbit AlexaFluor 555 (A31570, ThermoFisher, RRID: AB_2536180), anti-mouse AlexaFluor 555 (A31572, ThermoFisher, RRID: AB_162543), anti-goat AlexaFluor 488 (A11055, ThermoFisher, RRID: AB_2534102), and anti-rat AlexaFluor Plus 405 (A48268, ThermoFisher, RRID: AB_2890549). In some experiments, samples were counterstained in 300 nM DAPI (4′,6-diamidino-2-phenylindole; D1306, ThermoFisher), 20 μg ml⁻¹ AlexaFluor 488-labeled peanut (Arachis hypogaea) agglutinin lectin (PNA, L21409, Sigma), and/or 1:40 AlexaFluor 647 phalloidin (A22287, ThermoFisher) diluted in blocking buffer for 2 hours at room temperature, followed by 3 washes in PBS.

Kidneys were imaged in wells created with a 2 mm diameter biopsy punch in a ˜5 mm-thick layer of 15:1 (base: crosslinker) polydimethylsiloxane (PDMS) elastomer (Sylgard 184, 2065622, Ellsworth Adhesives) set in 35 mm coverslip-bottom dishes (FD35-100, World Precision Instruments). Imaging was performed using a Nikon Ti2-E microscope equipped with a CSU-W1 spinning disk (Yokogawa), a white light LED, laser illumination (100 mW 405, 488, and 561 nm lasers and a 75 mW 640 nm laser), a Prime 95B back-illuminated sCMOS camera (Photometrics), motorized stage, 4×/0.2 NA, 10×/0.25 NA and 20×/0.5 NA lenses (Nikon), and a stagetop environmental enclosure (OkoLabs).

Human iPSC-derived nephron progenitors. Nephron progenitor cells were generated from PENN123i-SV20 iPSCs (Induced Pluripotent Stem Cell Core Facility, University of Pennsylvania School of Medicine) or a SIX2^{EGFP} transgenic reporter iPSC line (SIX2-T2A-EGFP, Kidney Translational Research Center, Washington University Nephrology) (95) according to published protocols (66). Briefly, iPSCs were maintained in standard tissue-culture treated 24-well plates in stem cell maintenance medium plus supplement (mTeSR+kit, StemCell Technologies 100-0276) and 1× Penn/Strep from a 100× stock (Mediatech, MT30-002-C1). Cells were passaged using Accutase (StemCell Technologies, 07920) and plated at a density of 25,000 cells per well. Differentiation was induced with Advanced RPMI 1640 Medium (Fisher Scientific, 12-633-012), GlutaMAX (ThermoFisher, 35050061), 1× Penn/Strep and 7 μM CHIR 99021 (Tocris, 4423) either the next day or when cells reached 50-75% confluency. On day 4, media was changed to Advanced RPMI 1640 Medium, Glutamax, 1× Penn/Strep and 10 ng ml⁻¹ activin (R&D Systems, 338AC010CF). On Day 7 media was changed to Advanced RPMI 1640 Medium, Glutamax, 1× Penn/Strep and 10 ng ml⁻¹ FGF9 (R&D Systems, 273-F9-025). On day 9 (metanephric mesenchyme stage), cells were either 1) differentiated further in 10 ng ml⁻¹ FGF9 until day 21 with a 3 μM CHIR pulse over days 9-11, 2) dissociated and aggregated for use in spheroid experiments, or 3) cultured for up to 3 days in 10 ng ml⁻¹ FGF9 media to maintain them in the SIX2+ state.

2D iPSC-derived nephron progenitor immunofluorescence microscopy. Nephron progenitor cell 2D cultures were fixed in ice-cold 4% paraformaldehyde for 10 minutes. Fixed cells were then washed 2× in PBSG (DPBS+7.5 g/L glycine) 5 minutes each then washed once in DPBS. Cells were then permeabilized in 0.5% Triton-X-100 for 5 minutes at 4 C. Then cells were blocked for 1 hr at room temperature in IF Wash (DPBS+1 g/L Bovine Serum Albumin+0.2% Triton-X-100+0.04% Tween-20)+10% donkey serum. Cells were then incubated overnight at 4 C in appropriate dilution of primary antibody in IF Wash+10% donkey Serum. Cells were then washed 3× in IF wash at room temperature totaling 30 minutes then incubated in appropriate dilution of secondary antibody at room temperature in IF wash+10% donkey serum. Cells were again washed 3× in IF wash totaling 30 minutes. Samples were counterstained in 300 nM DAPI in DPBS and washed 1× in DPBS then imaged using confocal microscopy. Primary antibodies and dilutions included mouse anti-E-cadherin (clone 34, 1:200, 610404, BD Biosciences, RRID: AB_397787), rabbit anti-SLC12A1 (1:300, ab171747, Abcam, RRID: AB_2802126), goat anti-Gata3 (1:20, AF2605, R&D Systems, RRID: AB_2108571). Secondary antibodies (all raised in donkey) were used at 1:300 dilution and include anti-rabbit AlexaFluor 647 (A31573, ThermoFisher, RRID: AB_2536183), anti-goat AlexaFluor 488 (A11055, ThermoFisher, RRID: AB_2534102), and anti-mouse AlexaFluor 555 (A31572, ThermoFisher, RRID: AB_162543).

Validation of iPSC differentiation by qPCR. RNA extraction was performed on cells from individual wells of a 24-well tissue-culture dish at each differentiation time-point using an RNEasy mini kit (Qiagen 74104). cDNA was generated from RNA using an Applied Biosystems High Capacity cDNA Reverse Transcription kit (ThermoFisher 4368814), following the manufacturer protocol and using 2 ng of RNA in the reaction for each sample. Applied Biosystems Sybr Green PCR Master Mix (ThermoFisher 4344463) was used for qPCR in an Applied Biosystems 7300 thermocycler. Applied Biosystems suggested protocol for Sybr Green PCR Master Mix was followed for temperatures and times. 25 ng of cDNA were used in each reaction along with the appropriate qPCR primers at 300 nM. qPCR primers for Actin-Beta, OSRI, and PAX2 were used as previously described (61). DeltaDeltaCt values were calculated using ACTB as a housekeeping gene.

Nephron progenitor spheroid differentiation assays. Day 9 cap mesenchyme-stage nephron progenitors were lifted with Accutase and re-plated at 50,000 cells per well in 96-well ultra low attachment round-bottom plates (Corning 7007) in Advanced RPMI 1640 Medium, Glutamax, 1× Penn/Strep and 10 ng ml⁻¹ FGF9 (“basal media”) plus the specified perturbation conditions, which included 3 μM CHIR, 10 μM Y27632 (StemCell Technologies, 72304) from a 10 mM stock in DMSO, and/or 30 μM(S) (S)-(−)-blebbistatin (Tocris, 1852) from a 30 mM stock in DMSO. Cells were then aggregated by plate centrifugation at 300 g for 30 s. After 48 hr in culture, spheroids were collected and transferred to 1.7 mL tubes using a p200 pipette tip widened by cutting the tip to size with a razor blade. Excess media was removed and spheroids were fixed in ice-cold 4% paraformaldehyde in PBS for 15 min. Spheroids were then stained for immunofluorescence microscopy similar to kidneys (see Kidney immunofluorescence imaging.) via primary and secondary antibody incubation for 48 hr each at 4 C and washes in PBSTX totaling 6 hours. Primary antibodies and dilutions included mouse anti-Lhx1 (1:50, 4F2, Developmental Studies Hybridoma Bank, University of Iowa, RRID: AB_531784) and rabbit anti-Six2 (1:600, 11562-1-AP, Proteintech, RRID: AB_2189084). Spheroids were then incubated in 1 μg ml⁻¹ DAPI in PBS at 4 C for 1 hr before washing once in ice-cold PBS. Spheroids were transferred for imaging using a widened p200 pipette tip to a cover-slip bottom 24-well plate (Greiner 662892). Excess PBS was removed and spheroids were incubated with ˜20 μl of FocusClear (Cedarlane Labs, FC-101) for 20 min at room temperature prior to confocal imaging. Spheroids were imaged using 5 μm z-steps.

Quantification of spheroid cell differentiation. Maximum intensity projections of spheroids were generated and further processed in FIJI. DAPI signal was used to segment spheroids and calculate their areas in number of pixels. SIX2 and LHX1 signal was segmented using 50 pix rolling ball background subtraction and thresholding to remove background signal. The total numbers of SIX2+ and LHX1+ pixels were then divided by the total number of DAPI+ pixels to normalize positive pixels by spheroid area.

Embryonic kidney data annotation. Ureteric tip positions were manually annotated from confocal stacks in FIJI using the multi-point tool. Nephrons per tip were manually annotated from confocal stacks of the SIX2 and PNA channels, summing across SIX2+ pre-tubular aggregates/early renal vesicles and later stages for which connecting tubule junctions with ureteric tips were visible as circular intersections in the PNA channel at tips.

Confocal immunofluorescence stacks on the SIX2 channel were manually annotated to extract kidney outlines at each z plane by manually tracing in FIJI on an Apple iPad. These outlines constituted a binary mask stack used for input for curvature analysis.

Voronoi, shape index, and defect charge analysis. Delaunay triangulations and Voronoi diagrams were created from ureteric tip coordinates in MATLAB, roughly filtered by polygon area and manually edited to remove edge cells. The coordination number was determined as the number of sides of each Voronoi cell, and shape index p was determined via p=P/√A, where P is the perimeter of a Voronoi cell and A is its area. Heat maps of shape index were produced by interpolation of shape index data in MATLAB using the ‘v4’ method in griddata.m. The total disclination defect charge Q for a particular Voronoi diagram was determined by summing the topological charges s over all Voronoi cells (note: s=6−z, where z is the coordination number of a cell).

Packing model. We used physics-based modeling in the Rhino (v7, Robert McNeel & Associates) Grasshopper Kangaroo2 environment (Daniel Piker) to demonstrate qualitative aspects of repulsive sphere packing on flat surfaces and large spheres in FIG. 1B for schematic purposes only. Quantitative principles of this software environment are detailed in (17). We used the SphereCollide goal to model sphere mutual repulsion and the OnMesh goal to hold spheres at flat or larger spherical interfaces, each with arbitrarily high energy potential weightings to mimic previously described features of particle packings on surfaces (18-20,96). To create overlay schematics, sphere coordinates were exported from Rhino and used as input to the same Voronoi and coordination number analysis performed in MATLAB.

Kidney curvature analysis. 3D mask stacks of kidney outlines were first exported from 3D viewer in FIJI as binary.stl meshes representing kidney surfaces (FIG. 7). These .stl files were then imported into Rhino, remeshed down to ˜1,000 polygons using quad remesh, smoothed, triangulated, draped, and split to remove the bottom face. Contour cross-sections were created at 20 μm increments along the long axis and lofted to create a smooth, regular mesh representing the outer kidney surface. Surface meshes were exported as .stl files, imported into MATLAB and represented as height maps using stlread and trisurf commands. Exported meshes were also analyzed to determine the Gaussian curvature local to each polygon via a custom workflow in Rhino Grasshopper. The area-weighted sum of polygon curvature, I=∫_{A}G dA over an equivalent area covered by the Voronoi diagram for that surface was then used to compute the integrated Gaussian curvature in units of disclinations,

$$Ω = \frac{1}{π / 3} .$$

Rigidity analysis. We describe close-packed tip domains using a 2D cell vertex model (38,40,97,98), representing the mechanical energy of each tip domain by:

$${E}_{i} = {K ( {A}_{i} - {A}_{0} )}^{2} + ξ {P}_{i}^{2} + γ {P}_{i}$$

Where the first term represents collective cell elasticity (stiffness) in tip domains, a product of a stiffness constant and actual and preferred domain cross-sectional areas respectively. The second term represents collective cell contractility in tip domains, a product of a constant and the square of the cell perimeter, and the third term represents an interfacial line tension proportional to cell perimeter, set by competition between stroma contractility and cell-cell adhesion at the cap mesenchyme-stroma boundary (FIG. 5A). These terms are analogous to cell elasticity, cortical contractility, and interfacial tension terms for confluent monolayers. These energy terms are justified firstly by macroscopic behavior of embryonic kidney cortex explants, which actively contract and round up without loss of tip domain adhesion to neighbors in the hours after cutting (17). Secondly, elasticity of tip domains is implied by positive stiffness values returned by microindentation on a length-scale appropriate to tip domain extents in xy and z (FIG. 3D), and interfacial line tensions are implied by rebound velocities being positive in domain-domain interface ablation experiments (FIG. 5F).

Several lines of analysis predict a rigidity transition in cell/uncoupled tip domain systems governed by the above energy equation at a critical shape index of p*=3.81, the simplest of which is isostaticity (degree of freedom analysis) (38). Bi et al. began from Euler's formula for polygons tiling the 2D plane with periodic boundary conditions: 0=V−E+M, where E is the number of edges, V is the number of vertices, and M is the number of cells (38). Since two cells share each edge, Mz=2E, where z is the coordination number. These two expressions give V=M(z/2−1). The degrees of freedom n_{dof}=2V, accounting for translation of each vertex in 2D. Three constraints per cell are needed for rigidity (to achieve a force balance constraining 2D translation and a torque balance constraining rotation), i.e. n_{c}=3M. At isostaticity (rigidity), n_{dof}=n_{c}, i.e. 2V=3M. Substituting into Euler's formula then gives zᵢₛₒ=5. This suggests that rigidity will occur when the average shape index drops below the shape index of a pentagon=p*≈3.81.

However, for coupled tip domains in which daughter tubules share a physical linkage through their parent node, we note that only two constraints rather than three are needed for one of each couple, since one translational constraint is accounted for by the physical linkage. This makes n_{c}=2.5M and at isostaticity, 4V=5M, giving zᵢₛₒ=4.5. Rigidity should therefore occur when the average shape index drops below that for an equal mixture of squares and pentagons, implying p*=(4+3.81)/2≈3.91.

Spatial autocorrelation analysis. 2D spatial autocorrelation heatmaps of tip regions were computed from maximum projections of confocal z stacks on the PNA fluorescence channel using the Wiener-Khintchine theorem in MATLAB (autocorr2d.m, Tristan Ursell).

Microindentation. Free-standing explanted mouse kidneys were micro-indented using established methods (43). Instrumentation was the same as described by Levental et al., except indenter arm height was controlled by a stepper motor (L4018S1204-M6, Nanotec, Munich) rather than a hydraulic micromanipulator. The indenter was fabricated from cylindrical 30 gauge (AWG) SAE 316L stainless steel wire having a diameter of 255 μm. Briefly, after calibrating the spring constant of the tensiometric sensor, each kidney was placed in a 35 mm culture dish and bulk media wicked away to leave the kidney surface semi-dry. The indenter was immediately lowered at a rate of 12.5 μm s⁻¹ using the z-stage of the instrument to an indentation depth of ˜30 μm into the kidney surface while automatically recording time, force on the indenter, and indenter z position. Indentation was then halted and force vs. time recorded for an additional 60-120 s. Stiffness was inferred using the following relationship for indentation of a soft homogeneous material by a flat hard surface:

$${E}_{t i s s u e} = \frac{{F}_{appl} ( 1 - {ν}_{tissue}^{2} )}{{h}_{i n d} \cdot κ \cdot 2 r}$$

Where Fₐₚₚₗ/h_{ind} is the slope of force vs. indentation depth from the point of contact after accounting for sensor spring constant. v is the Poisson ratio of the tissue, assumed to be 0.5, κ is the Hayes correction factor (99), accounting for finite sample thickness (κ was determined for each stage based on average kidney width inferred from confocal image stacks), and 2r is the diameter of the probe (255 μm).

Similar to previous analysis (43), force vs. time (1) traces at fixed indentation depth were normalized to maximum force and fit to the following three-parameter relaxation relationship using non-linear least-squares fitting in MATLAB:

$${F}_{norm} = 1 - ( 1 - {G}_{∞} ) ( 1 - {e}^{- t / τ} ) - kt$$

This relationship implies a Kelvin-Voigt material with an extra viscous damper in series, where G_{∞} and τ are an equilibrium modulus and relaxation time constant, respectively. We refer to k as a viscous relaxation constant in this work.

Force inference. Bayesian force inference was applied according to a recently published procedure that infers maps of tissue tension and pressure based on observed variations in edge lengths and angles at cell vertices, assuming that tissues are in instantaneous mechanical equilibrium, tissue mechanics are dominated by in-plane tensions and pressures, and that interfacial tensions are positive (49,51). The Bayesian approach is appropriate here, since it is less reliant on accurate measurement of interface curvatures, making it ideal for cases with large numbers of cells and small interfacial curvatures (51). Voronoi diagrams were first processed in the Tissue Analyzer plugin in FIJI (100), and segmentation output data were then passed to MATLAB for Bayesian force inference using code published by Kong et al. (51), which was adapted from earlier work by Ishihara et al. (49). We used this code to compute stress tensors and their isotropic and anisotropic (deviatoric) components for each tip domain using Batchelor's formula (51). Since stress outputs are relative here, we normalized them to the largest median stress in a given set of comparisons.

Laser ablation. E17 kidney explants were labeled in 20 μg ml-1 Alexa Fluor 488-PNA and 1:1000 CellTracker Red (ThermoFisher C34552, 10 mM stock in DMSO) in Dulbecco's minimum essential medium (DMEM, 10-013-CV, Corning) for 1 hour at 37° C., washed 3× in DMEM and placed in 2 mm-diameter PDMS wells (see Kidney immunofluorescence imaging), this time plasma bonded to a quartz coverslip (No. 1 thickness, Ted Pella, 26014) to maximize UV transmission. Kidneys were ablated using a 355 nm UV laser (Molecular Machines & Industries, SL uCut v1.0) through a 10× objective on a Nikon Ti2 controlled by MMI software. Cut opening time-lapses were imaged from the microscope monitor using an iPhone 8 video camera and spatially calibrated using on-screen fiducial marks in the MMI software. Kidneys were fixed after ablations in ice cold 4% PFA for 15 min, washed 3× with PBS and imaged by confocal microscopy (see Kidney immunofluorescence imaging).

scRNA-seq analysis. Gene expression matrices generated from scRNA-seq of dissociated E15.5 mouse embryonic kidneys in Lawlor et al. were used (24). These matrices were accessed from the Gene Expression Omnibus (GEO, NCBI) under accession code GSE118486 and sample mk1 was used for further analysis. Analysis was done similarly to Lawlor et al. In short, the Seurat library (101.102) was used to import the gene expression matrix to R and for downstream analyses. Cells with less than 200 genes expressed or with greater than 7.5% mitochondrial gene expression were removed. Genes expressed in less than 3 cells were removed from the dataset. The filtered dataset contained 2,708 cells with an average of 2,441 unique genes detected per cell. Cell cycle scoring was done using the CellCycleScoring function. Scaled data matrices were then generated using the ScaleData function. SNN clustering was performed for whole kidney data using resolution 0.5 for the first 15 principal components calculated from a set of 2000 variable genes. Differentially expressed marker genes were identified using a Wilcoxon rank sum test and compared to the published list of marker genes from Lawlor et al. to assign cell identity. SNN clustering was then performed on cells belonging to the nephron lineage clusters using resolution 0.5 for the first 12 principal components calculated from a set of 2000 variable genes. Again, differentially expressed marker genes were identified in Seurat using Wilcoxon rank sum test and compared to a list of marker genes for cells within the nephron lineage cluster published by Lawlor et al. to assign cell identity. Fold-change and Wilcoxon rank sum p-value for individual genes in volcano plots were calculated by comparing the committing cluster to the progenitor cell clusters.

Gene Set Enrichment Analysis (GSEA) An unranked list of 171 highly upregulated genes in the committing cluster compared to the progenitor cell cluster was generated by taking genes with log2 (fold-change) of at least 0.25 and Bonferroni corrected adjusted Wilcoxon rank sum test p-value less than 0.05 between the clusters. The top 10most statistically significant overrepresented Gene Ontology terms in this gene set were found using the Gene Set Enrichment Analysis (GSEA) online software (103), to compare this gene list to Gene Ontology (GO) gene sets (58). Gene set enrichment plots were generated using GSEA desktop software. All genes expressed in at least 3 cells were ranked using log2 (fold-change) of average expression in the committing cluster against the progenitor clusters in Lawlor et al. to compare against the specified gene sets (24).

Statistical analysis. One-way analysis of variance (ANOVA) with correction for multiple comparisons using Tukey's honestly significant difference test was performed in MATLAB using anoval.m and multcompare.m functions.

### REFERENCES

1. Rumballe BA, Georgas KM, Combes AN, Ju AL, Gilbert T, Little MH. Nephron formation adopts a novel spatial topology at cessation of nephrogenesis. Dev Biol. 2011 Dec 1;360 (1):110-22.
2. Pohl M, Bhatnagar V, Mendoza SA, Nigam SK. Toward an etiological classification of developmental disorders of the kidney and upper urinary tract. Kidney Int. 2002 Jan;61(1):10-9.
3. Brenner BM, Mackenzie HS. Nephron mass as a risk factor for progression of renal disease. Kidney Int Suppl. 1997 Dec;63:S124-7.
4. Brenner BM, Garcia DL, Anderson S. Glomeruli and blood pressure. Less of one, more the other? Am J Hypertens. 1988 Oct;1(4 Pt 1):335-47.
5. Osathanondh V, Potter EL. DEVELOPMENT OF HUMAN KIDNEY AS SHOWN BY MICRODISSECTION. II. RENAL PELVIS, CALYCES, AND PAPILLAE. Arch Pathol. 1963 Sep;76:277-89.
6. Lefevre JG, Short KM, Lamberton TO, Michos O, Graf D, Smyth IM, et al. Branching morphogenesis in the developing kidney is governed by rules that pattern the ureteric tree. Development. 2017 Dec 1;144(23):4377-85.
7. Carroll TJ, Park JS, Hayashi S, Majumdar A, McMahon AP. Wnt9b plays a central role in the regulation of mesenchymal to epithelial transitions underlying organogenesis of the mammalian urogenital system. Dev Cell. 2005 Aug;9(2):283-92.
8. Lu BC, Cebrian C, Chi X, Kuure S, Kuo R, Bates CM, et al. Etv4 and Etv5 are required downstream of GDNF and Ret for kidney branching morphogenesis. Nat Genet. 2009 Dec;41(12):1295-302.
9. Costantini F, Kopan R. Patterning a complex organ: branching morphogenesis and nephron segmentation in kidney development. Dev Cell. 2010 May 18;18(5):698-712.
10. Schuchardt A, D'Agati V, Larsson-Blomberg L, Costantini F, Pachnis V. Defects in the kidney and enteric nervous system of mice lacking the tyrosine kinase receptor Ret. Nature. 1994 Jan 27;367(6461):380-3.
11. Pichel JG, Shen L, Sheng HZ, Granholm AC, Drago J, Grinberg A, et al. Defects in enteric innervation and kidney development in mice lacking GDNF. Nature. 1996 Jul 4;382(6586):73-6.
12. Little MH. Returning to kidney development to deliver synthetic kidneys. Dev Biol. 2021 Jun;474:22-36.
13. Lindström NO, Lawrence ML, Burn SF, Johansson JA, Bakker ERM, Ridgway RA, et al. Integrated β-catenin, BMP, PTEN, and Notch signalling patterns the nephron. Elife. 2015 Feb 3;3:e04000.
14. Georgas K, Rumballe B, Valerius MT, Chiu HS, Thiagarajan RD, Lesieur E, et al. Analysis of early nephron patterning reveals a role for distal RV proliferation in fusion to the ureteric tip via a cap mesenchyme-derived connecting segment. Dev Biol. 2009 Aug 15;332(2):273-86.
15. Kao RM, Vasilyev A, Miyawaki A, Drummond IA, McMahon AP. Invasion of distal nephron precursors associates with tubular interconnection during nephrogenesis. J Am Soc Nephrol. 2012 Oct;23(10):1682-90.
16. Short KM, Combes AN, Lefevre J, Ju AL, Georgas KM, Lamberton T, et al. Global quantification of tissue dynamics in the developing mouse kidney. Dev Cell. 2014 Apr 28;29(2):188-202.
17. Prahl LS, Viola JM, Liu J, Hughes AJ. The developing kidney actively negotiates geometric packing conflicts to avoid defects [Internet]. bioRxiv. 2021 [cited 2022 Feb 26]. p. 2021.11.29.470441. Available from:
https://www.biorxiv.org/content/biorxiv/early/2021/12/01/2021.11.29.470441
18. Irvine WTM, Vitelli V, Chaikin PM. Pleats in crystals on curved surfaces. Nature. 2010 Dec 16;468(7326):947-51.
19. Bausch AR, Bowick MJ, Cacciuto A, Dinsmore AD, Hsu 4. M. F., Nelson DR, et al. Grain Boundary Scars and Spherical Crystallography. Science. 2003;299.
20. Jiménez FL, Stoop N, Lagrange R, Dunkel J, Reis PM. Curvature-Controlled Defect Localization in Elastic Surface Crystals. Phys Rev Lett. 2016 Mar 11;116(10):104301.
21. Brojan M, Terwagne D, Lagrange R, Reis PM. Wrinkling crystallography on spherical surfaces. Proc Natl Acad Sci U S A. 2015 Jan 6;112(1):14-9.
22. Bowick MJ, Giomi L. Two-dimensional matter: order, curvature and defects. Adv Phys. 2009 Sep 1;58(5):449-563.
23. Combes AN, Lefevre JG, Wilson S, Hamilton NA, Little MH. Cap mesenchyme cell swarming during kidney development is influenced by attraction, repulsion, and adhesion to the ureteric tip. Dev Biol. 2016 Oct 15;418(2):297-306.
24. Lawlor KT, Zappia L, Lefevre J, Park JS, Hamilton NA, Oshlack A, et al. Nephron progenitor commitment is a stochastic process influenced by cell migration. Elife [Internet]. 2019 Jan 24;8. Available from: http://dx.doi.org/10.7554/eLife.41156
25. Wilson SB, Little MH. The origin and role of the renal stroma. Development [Internet]. 2021 Oct 1;148(19). Available from:
http://dx.doi.org/10.1242/dev.199886
26. Davies JA, Hohenstein P, Chang CH, Berry R. A self-avoidance mechanism in patterning of the urinary collecting duct tree. BMC Dev Biol. 2014 Sep 10;14:35.
27. England AR, Chaney CP, Das A, Patel M, Malewska A, Armendariz D, et al. Identification and characterization of cellular heterogeneity within the developing renal interstitium. Development [Internet]. 2020 Aug 14;147(15). Available from: http://dx.doi.org/10.1242/dev.190108
28. Munro DAD, Hohenstein P, Davies JA. Cycles of vascular plexus formation within the nephrogenic zone of the developing mouse kidney. Sci Rep. 2017 Jun. 12;7(1):3273.
29. Yao Z. Stress-induced ordering of two-dimensional packings of elastic spheres. Phys Rev E. 2020 Jun;101(6-1):062904.
30. Princen HM. Rheology of foams and highly concentrated emulsions. II. experimental study of the yield stress and wall effects for concentrated oil-in-water emulsions. J Colloid Interface Sci. 1985 May 1;105(1):150-71.
31. Princen HM. Rheology of foams and highly concentrated emulsions: I. Elastic properties and yield stress of a cylindrical model system. J Colloid Interface Sci. 1983 Jan 1;91(1):160-75.
32. Cohen-Addad S, Höhler R, Pitois O. Flow in Foams and Flowing Foams. Annu Rev Fluid Mech. 2013 Jan 3;45(1):241-67.
33. Mason TG, Bibette J, Weitz DA. Yielding and flow of monodisperse emulsions. J Colloid Interface Sci. 1996 May; 179(2):439-48.
34. Mongera A, Rowghanian P, Gustafson HJ, Shelton E, Kealhofer DA, Carn EK, et al. A fluid-to-solid jamming transition underlies vertebrate body axis elongation. Nature. 2018 Sep;561(7723):401-5.
35. Blumenfeld R. Disorder Criterion and Explicit Solution for the Disc Random Packing Problem. Phys Rev Lett. 2021 Sep 10;127(11):118002.
36. Bolton F, Weaire D. Rigidity loss transition in a disordered 2D froth. Phys Rev Lett. 1990 Dec 31;65(27):3449-51.
37. Bi D, Lopez JH, Schwarz JM, Manning ML. Energy barriers and cell migration in densely packed tissues. Soft Matter. 2014 Mar 28;10(12):1885-90.
38. Bi D, Lopez JH, Schwarz JM, Manning ML. A density-independent rigidity transition in biological tissues. Nat Phys. 2015 Sep 21;11(12):1074-9.
39. Park JA, Kim JH, Bi D, Mitchel JA, Qazvini NT, Tantisira K, et al. Unjamming and cell shape in the asthmatic airway epithelium. Nat Mater. 2015 Oct;14(10):1040-8.
40. Farhadifar R, Röper JC, Aigouy B, Eaton S, Jülicher F. The influence of cell mechanics, cell-cell interactions, and proliferation on epithelial packing. Curr Biol. 2007 Dec 18;17(24):2095-104.
41. Liu AJ, Nagel SR. The Jamming Transition and the Marginally Jammed Solid. Annu Rev Condens Matter Phys. 2010 Aug 10;1(1):347-69.
42. Broedersz CP, Mao X, Lubensky TC, Mackintosh FC. Criticality and isostaticity in fibre networks. Nat Phys. 2011 Oct 30;7(12):983-8.
43. Levental I, Levental KR, Klein EA, Assoian R, Miller RT, Wells RG, et al. A simple indentation device for measuring micrometer-scale tissue stiffness. J Phys Condens Matter. 2010 May 19;22(19):194120.
44. Muncie JM, Ayad NME, Lakins JN, Xue X, Fu J, Weaver VM. Mechanical Tension Promotes Formation of Gastrulation-like Nodes and Patterns Mesoderm Specification in Human Embryonic Stem Cells. Dev Cell. 2020 Dec 21;55(6):679-94.e11.
45. Shyer A E, Rodrigues AR, Schroeder GG, Kassianidou E, Kumar S, Harland RM. Emergent cellular self-organization and mechanosensation initiate follicle pattern in the avian skin. Science. 2017 Aug 25;357(6353):811-5.
46. Parada C, Banavar SP, Khalilian P, Rigaud S, Michaut A, Liu Y, et al. Mechanical feedback defines organizing centers to drive digit emergence. Dev Cell. 2022 Apr 11;57(7):854-66.e6.
47. Reginensi A, Scott RP, Gregorieff A, Bagherie-Lachidan M, Chung C, Lim DS, et al. Yap-and Cdc42-dependent nephrogenesis and morphogenesis during mouse kidney development. PLOS Genet. 2013 Mar;9(3):e1003380.
48. Little MH, McMahon AP. Mammalian kidney development: principles, progress, and projections. Cold Spring Harb Perspect Biol [Internet]. 2012 May 1;4(5). Available from: http://dx.doi.org/10.1101/cshperspect.a008300
49. Ishihara S, Sugimura K. Bayesian inference of force dynamics during morphogenesis. J Theor Biol. 2012 Nov 21;313:201-11.
50. Guirao B, Rigaud SU, Bosveld F, Bailles A, López-Gay J, Ishihara S, et al. Unified quantitative characterization of epithelial tissue development. Elife [Internet]. 2015 Dec 12;4. Available from: http://dx.doi.org/10.7554/eLife.08519
51. Kong W, Loison O, Chavadimane Shivakumar P, Chan EH, Saadaoui M, Collinet C, et al. Experimental validation of force inference in epithelia from cell to tissue scale. Sci Rep. 2019 Oct 10;9(1):14647.
52. Janmey PA, Wells RG, Assoian RK, McCulloch CA. From tissue mechanics to transcription factors. Differentiation. 2013 Oct;86(3):112-20.
53. Muncie JM, Weaver VM. The Physical and Biochemical Properties of the Extracellular Matrix Regulate Cell Fate. Curr Top Dev Biol. 2018 Mar 21;130:1-37.
54. Hayward MK, Muncie JM, Weaver VM. Tissue mechanics in stem cell fate, development, and cancer. Dev Cell. 2021 Jul 12;56(13):1833-47.
55. Vining KH, Mooney DJ. Mechanical forces direct stem cell behaviour in development and regeneration. Nat Rev Mol Cell Biol. 2017 Dec;18 (12):728-42.
56. Priya R, Allanki S, Gentile A, Mansingh S, Uribe V, Maischein HM, et al. Tension heterogeneity directs form and fate to pattern the myocardial wall. Nature. 2020 Dec;588(7836):130-4.
57. De Belly H, Stubb A, Yanagida A, Labouesse C, Jones PH, Paluch EK, et al. Membrane Tension Gates ERK-Mediated Regulation of Pluripotent Cell Fate. Cell Stem Cell. 2021 Feb 4;28(2):273-84.e6.
58. Ashburner M, Ball CA, Blake JA, Botstein D, Butler H, Michael Cherry J, et al. Gene Ontology: tool for the unification of biology [Internet]. Vol. 25, Nature Genetics. 2000. p. 25-9. Available from: http://dx.doi.org/10.1038/75556
59. Przybyla L, Muncie JM, Weaver VM. Mechanical Control of Epithelial-to-Mesenchymal Transitions in Development and Cancer. Annu Rev Cell Dev Biol. 2016 Oct 6;32:527-54.
60. Belinky F, Nativ N, Stelzer G, Zimmerman S, Iny Stein T, Safran M, et al. PathCards: multi-source consolidation of human biological pathways. Database [Internet]. 2015 Feb 27;2015. Available from: http://dx.doi.org/10.1093/database/bav006
61. Morizane R, Lam AQ, Freedman BS, Kishi S, Valerius MT, Bonventre JV. Nephron organoids derived from human pluripotent stem cells model kidney development and injury. Nat Biotechnol. 2015 Nov;33(11):1193-200.
62. Takasato M, Er PX, Chiu HS, Maier B, Baillie GJ, Ferguson C, et al. Kidney organoids from human iPS cells contain multiple lineages and model human nephrogenesis. Nature. 2015 Oct 22;526(7574):564-8.
63. Combes AN, Zappia L, Er PX, Oshlack A, Little MH. Single-cell analysis reveals congruence between kidney organoids and human fetal kidney. Genome Med. 2019 Jan 23;11(1):3.
64. Lindström NO, Guo J, Kim AD, Tran T, Guo Q, De Sena Brandine G, et al. Conserved and Divergent Features of Mesenchymal Progenitor Cell Types within the Cortical Nephrogenic Niche of the Human and Mouse Kidney. J Am Soc Nephrol. 2018 Mar;29(3):806-24.
65. Wu H, Uchimura K, Donnelly EL, Kirita Y, Morris SA, Humphreys BD. Comparative Analysis and Refinement of Human PSC-Derived Kidney Organoid Differentiation with Single-Cell Transcriptomics. Cell Stem Cell. 2018 Dec 6;23(6):869-81.e8.
66. Morizane R, Bonventre JV. Generation of nephron progenitor cells and kidney organoids from human pluripotent stem cells. Nat Protoc. 2017 Jan;12(1):195-207.
67. Park JS, Ma W, O'Brien LL, Chung E, Guo JJ, Cheng JG, et al. Six2 and Wnt regulate self-renewal and commitment of nephron progenitors through shared gene regulatory networks. Dev Cell. 2012 Sep 11;23(3):637-51.
68. Park JS, Valerius MT, McMahon AP. Wnt/beta-catenin signaling regulates nephron induction during mouse kidney development. Development. 2007 Jul;134(13):2533-9.
69. Brown AC, Muthukrishnan SD, Guay JA, Adams DC, Schafer DA, Fetting JL, et al. Role for compartmentalization in nephron progenitor differentiation. Proc Natl Acad Sci U S A. 2013 Mar 19;110(12):4640-5.
70. Motamedi FJ, Badro DA, Clarkson M, Lecca MR, Bradford ST, Buske FA, et al. WT1 controls antagonistic FGF and BMP-pSMAD pathways in early renal progenitors. Nat Commun. 2014 Jul 17;5:4444.
71. Essafi A, Webb A, Berry RL, Slight J, Burn SF, Spraggon L, et al. A wt1-controlled chromatin switching mechanism underpins tissue-specific wnt4 activation and repression. Dev Cell. 2011 Sep 13;21(3):559-74.
72. Volovelsky O, Nguyen T, Jarmas A E, Combes AN, Wilson SB, Little MH, et al. Hamartin regulates cessation of mouse nephrogenesis independently of Mtor. Proc Natl Acad Sci U S A. 2018 Jun 5;115(23):5998-6003.
73. Oxburgh L. Growth control of the kidney. Curr Top Dev Biol. 2022 Feb 28;148:237-63.
74. Cebrian C, Asai N, D'Agati V, Costantini F. The number of fetal nephron progenitor cells limits ureteric branching and adult nephron endowment. Cell Rep. 2014 Apr 10;7(1):127-37.
75. Combes AN, Wilson S, Phipson B, Binnie BB, Ju A, Lawlor KT, et al. Haploinsufficiency for the Six2 gene increases nephron progenitor proliferation promoting branching and nephron number. Kidney Int. 2018 Mar;93(3):589-98.
76. Ramalingam H, Fessler AR, Das A, Valerius MT, Basta J, Robbins L, et al. Disparate levels of beta-catenin activity determine nephron progenitor cell fate. Dev Biol. 2018 Aug 1;440(1):13-21.
77. Karner CM, Das A, Ma Z, Self M, Chen C, Lum L, et al. Canonical Wnt9b signaling balances progenitor cell expansion and differentiation during kidney development. Development. 2011 Apr;138(7):1247-57.
78. Blank U, Brown A, Adams DC, Karolak MJ, Oxburgh L. BMP7 promotes proliferation of nephron progenitor cells via a JNK-dependent mechanism. Development. 2009 Nov;136(21):3557-66.
79. Das A, Tanigawa S, Karner CM, Xin M, Lum L, Chen C, et al. Stromal-epithelial crosstalk regulates kidney progenitor cell differentiation. Nat Cell Biol. 2013 Sep;15(9):1035-44.
80. Fetting JL, Guay JA, Karolak MJ, Iozzo RV, Adams DC, Maridas DE, et al. FOXDI promotes nephron progenitor differentiation by repressing decorin in the embryonic kidney. Development. 2014 Jan;141 (1):17-27.
81. Mao Y, Francis-West P, Irvine KD. Fat4/Dchsl signaling between stromal and cap mesenchyme cells influences nephrogenesis and ureteric bud branching. Development. 2015 Aug 1;142(15):2574-85.
82. Brown AC, Muthukrishnan SD, Oxburgh L. A synthetic niche for nephron progenitor cells. Dev Cell. 2015 Jul 27;34(2):229-41.
83. Manoharan VN. Colloidal matter: Packing, geometry, and entropy. Science. 2015 Aug 28;349(6251):1253751.
84. Bowick MJ, Nelson DR, Travesset A. Interacting topological defects on frozen topographies. Phys Rev B Condens Matter. 2000 Oct 1;62(13):8738-51.
85. Sebinger DDR, Unbekandt M, Ganeva VV, Ofenbauer A, Werner C, Davies JA. A novel, low-volume method for organ culture of embryonic kidneys that allows development of cortico-medullary anatomical organization. PLOS One. 2010 May 10;5(5):e10550.
86. Lawlor KT, Vanslambrouck JM, Higgins JW, Chambon A, Bishard K, Arndt D, et al. Cellular extrusion bioprinting improves kidney organoid reproducibility and conformation. Nat Mater. 2021 Feb;20(2):260-71.
87. Dupont S, Morsut L, Aragona M, Enzo E, Giulitti S, Cordenonsi M, et al. Role of YAP/TAZ in mechanotransduction. Nature. 2011 Jun 8;474(7350):179-83.
88. Panciera T, Azzolin L, Cordenonsi M, Piccolo S. Mechanobiology of YAP and TAZ in physiology and disease. Nat Rev Mol Cell Biol. 2017 Dec;18(12):758-70.
89. Lindström NO, Hohenstein P, Davies JA. Nephrons require Rho-kinase for proximal-distal polarity development. Sci Rep. 2013 Sep 18;3(1):1-8.
90. Recuenco MC, Ohmori T, Tanigawa S, Taguchi A, Fujimura S, Conti MA, et al. Nonmuscle myosin II regulates the morphogenesis of metanephric mesenchyme-derived immature nephrons. J Am Soc Nephrol. 2015 May;26(5):1081-91.
91. Wanek N, Muneoka K, Holler-Dinsmore G, Burton R, Bryant SV. A staging system for mouse limb development. J Exp Zool. 1989 Jan;249(1):41-9.
92. Barak H, Boyle SC. Organ culture and immunostaining of mouse embryonic kidneys. Cold Spring Harb Protoc. 2011 Jan 1;2011(1):db.prot5558.
93. Combes AN, Short KM, Lefevre J, Hamilton NA, Little MH, Smyth IM. An integrated pipeline for the multidimensional analysis of branching morphogenesis. Nat Protoc. 2014 Dec;9(12):2859-79.
94. O'Brien LL, Combes AN, Short KM, Lindström NO, Whitney PH, Cullen-McEwen LA, et al. Wnt11 directs nephron progenitor polarity and motile behavior ultimately determining nephron endowment. Elife [Internet]. 2018 Dec 5;7. Available from: http://dx.doi.org/10.7554/eLife.40392
95. Vanslambrouck JM, Wilson SB, Tan KS, Soo JYC, Scurr M, Spijker HS, et al. A Toolbox to Characterize Human Induced Pluripotent Stem Cell-Derived Kidney Cell Types and Organoids. J Am Soc Nephrol. 2019 Oct;30(10):1811-23.
96. Negri C, Sellerio AL, Zapperi S, Miguel MC. Deformation and failure of curved colloidal crystal shells. Proc Natl Acad Sci U S A. 2015 Nov 24;112(47):14545-50.
97. Fletcher AG, Osterfield M, Baker RE, Shvartsman SY. Vertex models of epithelial morphogenesis. Biophys J. 2014 Jun 3;106(11):2291-304.
98. Nagai T, Honda H. A dynamic cell model for the formation of epithelial tissues. Philos Mag B. 2001 Jul 1;81(7):699-719.
99. Hayes WC, Keer LM, Herrmann G, Mockros LF. A mathematical analysis for indentation tests of articular cartilage. J Biomech. 1972 Sep;5(5):541-51.
100. Aigouy B, Umetsu D, Eaton S. Segmentation and Quantitative Analysis of Epithelial Tissues. Methods Mol Biol. 2016;1478:227-39.
101. Satija R, Farrell JA, Gennert D, Schier AF, Regev A. Spatial reconstruction of single-cell gene expression data. Nat Biotechnol. 2015 May;33(5):495-502.
102. Butler A, Hoffman P, Smibert P, Papalexi E, Satija R. Integrating single-cell transcriptomic data across different conditions, technologies, and species. Nat Biotechnol. 2018 Jun;36(5):411-20.
103. Subramanian A, Tamayo P, Mootha VK, Mukherjee S, Ebert BL, Gillette MA, et al. Gene set enrichment analysis: a knowledge-based approach for interpreting genome-wide expression profiles. Proc Natl Acad Sci U S A. 2005 Oct 25;102(43):15545-50.

### ASPECTS

The following Aspects are illustrative only and do not limit the scope of the present disclosure or the appended claims.

Aspect 1. A method of modulating nephron differentiation, comprising: effecting a change in mechanical stress experienced by at least one nephron progenitor cell so as to give rise to cell differentiation and primitive nephron aggregate formation by the at least one nephron progenitor cell, the at least one nephron progenitor cell optionally being present in a patterned arrangement.

A mechanical stress can be, for example, a compression, a stretching, a twisting, and the like. Compression and stretching are considered suitable, but are not a requirement.

Nephron progenitor cells can be arranged in a pattern. For example, the cells can be arranged in a grid-type pattern. The cells can also be arranged in a honeycomb or honeycomb-like pattern. Cells can also be arranged in a non-patterned configuration, for example, a random arrangement of cells in which the cells are simply packed together. A given cell can be adhered to or otherwise attached. For example, a nephron progenitor cell can be adhered to an adjacent nephron progenitor cell. A nephron progenitor cell can be adhered to a substrate, for example, a cell culture flask or other container.

It is not, however, a requirement that a nephron progenitor cell be adhered to or otherwise attached. As an example, a nephron progenitor cell can be placed between two surfaces, and relative motion of at least one surface can give rise to a compression and/or stretching of the cell; the relative motion of the at least one surface can, in some instances, act to roll the cell.

The primitive nephron aggregates can be further processed. The aggregates can be implanted or otherwise introduced, for example, to a subject or to an organoid or other tissue. Such implantation can be in vivo, but can also be in vitro. The methods can be performed in vivo, but can also be performed in vitro. The methods can be performed in connection with 3D tissues and/or 3D organoids, but can also be performed in connection with 2D monolayers.

Aspect 2. The method of Aspect 1, wherein the change in mechanical stress comprises changing application of an extrinsic mechanical stress experienced by the at least one nephron progenitor cell. The application of the extrinsic mechanical stress can be repeated or even periodic in nature, although this is not a requirement. The application of the extrinsic mechanical stress can be an application of the same stress, for example, a repeated stretching. The application of the extrinsic mechanical stress can also be application of different stresses, for example, the application of a compression followed by application of a stretching.

Aspect 3. The method of Aspect 2, wherein the extrinsic mechanical stress comprises one or more of an external compression, an external sliding force, an external stretching force, a vibration, and a sonication, changing environmental stiffness, or imparting boundary curvature.

Aspect 4. The method of any one of Aspects 1-3, wherein the change in mechanical stress comprises changing application of an intrinsic mechanical stress experienced by the at least one nephron progenitor cell.

Aspect 5. The method of Aspect 4, wherein changing application of the intrinsic mechanical stress comprises at least one of (i) effecting an optogenetic process, optionally in a discontinuous manner, and (ii) application of an active agent (for example, a chemical and/or biochemical agent), optionally in a discontinuous manner, or (iii) overexpression of an RNA or protein activator or inhibitor. Any one or more of the foregoing can be applied in a periodic manner, but this is not a requirement, as any one or more of the foregoing can be applied in a non-periodic manner. The intrinsic mechanical stress can include, for example, changing at least one of cell adhesion and cell tension. Intrinsic stress can be changed by, for example, effecting influx or efflux of one or more cellular contents.

Aspect 6. The method of Aspect 4, wherein changing application of the intrinsic mechanical stress comprises changing at least one of cell adhesion and cell tension.

Aspect 7. The method of Aspect 4, wherein changing application of the intrinsic mechanical stress comprises modulating at least one of a Wnt/β-catenin pathway, a Rho/ROCK pathway, a BMP/pSMAD pathway, a Yap/Taz pathway, a Notch pathway, a non-canonical Wnt pathway, stretch-activated ion channels/Ca2* signaling, or a MAPK pathway of the nephron progenitor cell.

Aspect 8. The method of any one of Aspects 1-7, wherein the change in mechanical stress is effected in a discontinuous manner, the discontinuous manner optionally being periodic.

Aspect 9. The method of any one of Aspects 1-7, wherein the change in mechanical stress is effected such that the at least one nephron progenitor cell experiences alternating levels of mechanical stress. As but one example, the nephron progenitor cell can be cycled back-and-forth between a first stress level and a second stress level, for example between a stressless state and a stressed state.

Aspect 10. The method of any one of Aspects 1-9, wherein the at least one nephron progenitor cell contacts at least one of a hydrogel, a biological extracellular matrix, or a polymeric shape-change material. Matrigel™ (solubilized basement membrane matrix secreted by Engelbreth-Holm-Swarm (EHS) mouse sarcoma cells), collagen, and other like materials can be used. One can use Geltrex™ and other similar media.

Aspect 11. The method of any one of Aspects 1-10, wherein effecting a change in mechanical stress experienced by at least one nephron progenitor cell is effected by effecting a change in an intrinsic mechanical stress or an extrinsic mechanical stress of at least one accessory cell in mechanical communication with the at least one nephron progenitor cell, the mechanical communication optionally being through an intermediate medium. As a non-limiting example, this can also be achieved through cells not in direct contact. For example, an accessory cell can impart a stress on an external matrix, which stress is then felt (i.e., communicated to) a nephron progenitor cell.

Aspect 12. The method of Aspect 11, wherein the change in intrinsic mechanical stress is effected by performing an optogenetic process of the at least one accessory cell. Various optogenetic processes are known in the art and can be used to, for example, modulate cellular forces and mechanotransduction.

Aspect 13. The method of any one of Aspects 1-12, further comprising effecting a mesenchymal-epithelial transition of a cell of a cell aggregate to form at least one nephron.

Aspect 14. The method of Aspect 13, wherein the transition is effected by effecting a change in mechanical stress experienced by the cell of the cell aggregate.

Aspect 15. The method of Aspect 14, wherein the method is performed so as to effect different stresses in different locations of the cell aggregate.

Aspect 16. The method of any one of Aspects 1-15, wherein the method is performed to give rise to a plurality of nephrons in a predetermined location.

Aspect 17. The method of Aspect 16, wherein the plurality of nephrons are located in one or more of a module configured for fluid communication with a subject or configured as a diagnostic device. A diagnostic device can be, for example, a test bed that includes nephron cells for use in evaluating the effect of a drug or other compound on the nephron cells, for example, an organ-on-a-chip or other test bed.

Aspect 18. The method of Aspect 17, wherein the module is characterized as being at least a portion of a synthetic kidney.

Aspect 19. The method of Aspect 13, further comprising introducing the at least one nephron to a subject.

Aspect 20. A system, the system configured to perform the method of any one of Aspects 1-19.

Aspect 21. The system of Aspect 20, wherein the system comprises a manipulator configured to exert a mechanical stress on a nephron progenitor cell or a cell of a cell aggregate.

Aspect 22. The system of any one of Aspects 20-21, wherein the system comprises a supply of an agent that modulates at least one of a Wnt/B-catenin pathway, a Rho/ROCK pathway, a BMP/pSMAD pathway, a Yap/Taz pathway, a Notch pathway, a non-canonical Wnt pathway, stretch-activated ion channels/Ca²⁺ signaling, or a MAPK pathway of the nephron progenitor cell. Without being bound to any particular theory or embodiment, modulating a non-canonical Wnt pathway may be particularly useful in controlling a mesenchymal-epithelial transition.

Aspect 23. The system of any one of Aspects 20-22, further comprising a source of illumination configured to effect an optogenetic process within a nephron progenitor cell. Such sources of illumination can include, for example, an LED or other sources known to those of ordinary skill in the art.

Aspect 24. A synthetic kidney comprising a nephron formed according to the method of any one of Aspects 1-19. Such a synthetic kidney can be, for example, portable or otherwise wearable. The synthetic kidney can be configured for fluid communication with the user and can include, for example, a fluid handling train to communicate fluid from the user to the synthetic kidney as well to handle fluid that has passed through the synthetic kidney.

Aspect 25. A cartridge comprising a nephron formed according to the method of any one of Aspects 1-19, the cartridge being configured for installation in a synthetic kidney. Such a cartridge can be removeable/replaceable.

### SUMMARY

The disclosed technology solves aspects of a lack of control over the time and place of nephron formation in kidney organoids, and uncovers a fundamental biology of mechanical regulation of nephron formation, and engineers the mechanical microenvironment and internal mechanical and adhesion state of nephron progenitor cells to mimic this regulation. (Previous attempts at such manipulation have not utilized mechanics to increase nephron yield in kidney organoids.)

In a commercial setting the disclosed technology can be used to increase nephron yield and organization in kidney replacement tissues. These organoids can be grown from patient-derived cells. This technology can also used for more controlled nephron formation for disease modeling, personalized medicine, and nephron toxicity assays. Increases in nephron yield and organization allow for implantation and/or integration to adult kidney tissues. This enables wearable, implantable, or external biosynthetic kidney devices.

Micropatterning of iPSC-derived nephron progenitor cells and microdevices can, as explained, be used to generate controllable and transient stress and strain profiles in cell cultures. Material properties of 2D and 3D culture systems can be used to control the mechanical state of nephron progenitor cells. Optogenetic control of proteins involved in regulating the actin cytoskeleton can be introduced to control mechanical stress within cells. Timed and/or cyclic release of drugs that control cell mechanical state through interacting with the actin cytoskeleton directly, actin cytoskeleton regulatory proteins, or mechanosensory pathways can be used. Any of the above can also be effected by the use of an accessory cell population with controlled mechanical tension properties that exerts an external force on nephron progenitors.

1. A method of modulating nephron differentiation, comprising:
effecting a change in mechanical stress experienced by at least one nephron progenitor cell so as to give rise to cell differentiation and primitive nephron aggregate formation by the at least one nephron progenitor cell,
the at least one nephron progenitor cell optionally being present in a patterned arrangement.

2. The method of claim 1, wherein the change in mechanical stress comprises changing application of an extrinsic mechanical stress experienced by the at least one nephron progenitor cell.

3. The method of claim 2, wherein the extrinsic mechanical stress comprises one or more of an external compression, an external stretching force, a vibration, and a sonication, changing environmental stiffness, or imparting boundary curvature.

4. The method of claim 1, wherein the change in mechanical stress comprises changing application of an intrinsic mechanical stress experienced by the at least one nephron progenitor cell.

5. The method of claim 4, wherein changing application of the intrinsic mechanical stress comprises at least one of (i) effecting an optogenetic process, optionally in a discontinuous manner, and (ii) application of an active agent, optionally in a discontinuous manner, or (iii) overexpression of an RNA or protein activator or inhibitor.

6. The method of claim 4, wherein changing application of the intrinsic mechanical stress comprises changing at least one of cell adhesion and cell tension.

7. The method of claim 4, wherein changing application of the intrinsic mechanical stress comprises modulating at least one of a Wnt/β-catenin pathway, a Rho/ROCK pathway, a BMP/pSMAD pathway, a Yap/Taz pathway, a Notch pathway, a non-canonical Wnt pathway, stretch-activated ion channels/Ca²⁺ signaling, or a MAPK pathway of the nephron progenitor cell.

8. The method of claim 1, wherein the change in mechanical stress is effected in a discontinuous manner, the discontinuous manner optionally being periodic.

9. The method of claim 1, wherein the change in mechanical stress is effected such that the at least one nephron progenitor cell experiences alternating levels of mechanical stress.

10. The method of claim 1, wherein the at least one nephron progenitor cell contacts at least one of a hydrogel, a biological extracellular matrix, or a polymeric shape-change material.

11. The method of claim 1, wherein effecting a change in mechanical stress experienced by at least one nephron progenitor cell is effected by effecting a change in an intrinsic mechanical stress or an extrinsic mechanical stress of at least one accessory cell in mechanical communication with the at least one nephron progenitor cell, the mechanical communication optionally being through an intermediate medium.

12. The method of claim 11, wherein the change in intrinsic mechanical stress is effected by performing an optogenetic process of the at least one accessory cell.

13. The method of claim 1, further comprising effecting a mesenchymal-epithelial transition of a cell of a cell aggregate to form at least one nephron.

14. The method of claim 13, wherein the transition is effected by effecting a change in mechanical stress experienced by the cell of the cell aggregate.

15. The method of claim 14, wherein the method is performed so as to effect different stresses in different locations of the cell aggregate.

16. The method of claim 1, wherein the method is performed to give rise to a plurality of nephrons in a predetermined location.

17. The method of claim 16, wherein the plurality of nephrons are located in one or more of a module configured for fluid communication with a subject or configured as a diagnostic device.

18. The method of claim 17, wherein the module is characterized as being at least a portion of a synthetic kidney.

19. The method of claim 13, further comprising introducing the at least one nephron to a subject.

20. A system, the system configured to perform the method of claim 1.

21. The system of claim 20, wherein the system comprises a manipulator configured to exert a mechanical stress on a nephron progenitor cell or a cell of a cell aggregate.

22. The system of claim 20, wherein the system comprises a supply of an agent that modulates at least one of a Wnt/β-catenin pathway, a Rho/ROCK pathway, a BMP/pSMAD pathway, a Yap/Taz pathway, a Notch pathway, a non-canonical Wnt pathway, stretch-activated ion channels/Ca²⁺ signaling, or a MAPK pathway of the nephron progenitor cell.

23. The system of claim 20, further comprising a source of illumination configured to effect an optogenetic process within a nephron progenitor cell.

24. A synthetic kidney comprising a nephron formed according to the method of claim 1.

25. A cartridge comprising a nephron formed according to the method of claim 1, the cartridge being configured for installation in a synthetic kidney.
